(19) United States
(12) Patent Application Publication
(10) Pub. No.: US 2026/0242824 A1
(43) Pub. Date: Aug. 20, 2026

(54) ONCOLYTIC VIRUSES TO TREAT BRAIN CANCER

(71) Applicant: The Brigham and Women's Hospital, Inc., Boston, MA (US)

(72) Inventors: Camilo Faust Akl, Boston, MA (US); Federico Giovannoni, Boston, MA (US); Francisco J. Quintana, Boston, MA (US)

(21) Appl. No.: 19/160,933

(22) PCT Filed: Mar. 1, 2024
(86) PCT No.: PCT/US2024/018146

§ 371 Date: Aug. 29, 2025

Related U.S. Application Data
(60) Provisional application No. 63/449,216, filed on Mar. 1, 2023.

(51) Int. Cl. C12N 15/86; A61K 35/763; A61K 39/00; A61P 35/00; C07K 14/005; C07K 16/28

(52) U.S. Cl. CPC C12N 15/86; A61K 35/763; A61P 35/00; C07K 14/005; C07K 16/2875; A61K 2039/505; C07K 2317/622; C12N 2710/16622; C12N 2710/16632; C12N 2710/16643

(57) ABSTRACT

Described herein are compositions comprising engineered herpes simplex viruses (oHSV) comprising a sequence encoding a TRAIL-blocking monoclonal antibody and methods for use thereof in treating brain cancer, e.g., glioblastoma (GBM).

### CLAIM OF PRIORITY

This application claims the benefit of U.S. Provisional Patent Application Ser. No. 63/449,216, filed on Mar. 1, 2023. The entire contents of the foregoing are hereby incorporated by reference.

### TECHNICAL FIELD

Described herein are compositions comprising engineered herpes simplex viruses (oHSV) comprising a sequence encoding a tumor necrosis factor-related apoptosis-inducing ligand (TRAIL)-blocking monoclonal antibody or antigen-binding fragment thereof, and methods for use thereof in treating brain cancer, e.g., glioblastoma (GBM).

### BACKGROUND

Glioblastoma (GBM) is the most common form of primary brain cancer, making up around 40% of all brain tumor cases [1]. Despite the use of aggressive treatments, it remains one of the most fatal malignancies, and patients have seen little therapeutic improvement in at least 3 decades. Due to its rapid progression and often late diagnosis, the 5-year survival rate remains below 10% and therapy options are limited to surgical resection, radio- and chemotherapy [2,3]. High inter-patient variability, in addition to the genetic, epigenetic, and transcriptomic heterogeneity observed between individual tumor cell clones within the same tumor altogether form a significant obstacle for the development of universally effective therapies. Therapeutic interest in modulating the complex mixture of non-malignant cells known as the tumor microenvironment (TME) has increased, considering successful cancer immunotherapy in various organs. Local cell types in the brain TME have been shown to affect with cancer growth, and include astrocytes, microglia, neurons, and oligodendrocytes, but also infiltrating peripheral immune cells such as monocytes, T cells, neutrophils, dendritic cells, natural killer cells, and various other lymphoid or myeloid cell types [4,5]. Tumor-dependent changes within the myeloid compartment (TAM) have been profiled extensively using single-cell technologies, revealing heterogeneous activation states [6-10]. Astrocytes, however, have remained largely understudied at high resolution in the context of brain malignancies [4], despite emerging alongside microglia as key orchestrators of neuroinflammatory responses [11-13]. In particular, their functional heterogeneity in various CNS disease contexts indicates that astrocyte interactions with immune cell types provide relevant local tuning signals for immune responses in the TME [14, 15]. Astrocytes promote brain tumor growth through various mechanisms involving direct tumor-astrocyte communication [16] [17-21], highlighting the need to comprehensively profile the major compartments of the TME in tandem, and even applying techniques that capture cell-cell interactions [14].

### SUMMARY

Astrocytes are intimately linked into the immune modulatory processes that govern homeostasis and neuroinflammation in the central nervous system (CNS) and display significant functional heterogeneity. However, the distinct roles of specific astrocyte subsets within the glioblastoma (GBM) tumor microenvironment (TME) are unknown. We performed single-cell RNA sequencing of astrocytes in human and mouse GBM tissue, identifying multiple astrocyte subsets. The most highly expanded astrocyte subset expressed Tumor necrosis factor-related apoptosis-inducing ligand (TRAIL), a marker of immunosuppressive astrocytes previously observed in chronic autoinflammatory disease. An IFNγ-induced astrocyte subpopulation plays an anti-inflammatory role in the context of MS by inducing apoptosis of autoreactive T cells via TRAIL [11]. In vivo perturbation of this molecule significantly extended survival by alleviating apoptotic pressure on tumor-reactive T cells. Closer examination of this astrocyte subset revealed STAT3 as a regulator of its gene signature. We identified tumor-derived IL-11 as a central molecule involved in the induction of STAT3 signaling and TRAIL expression in astrocytes. The IL-11-TRAIL axis was therapeutically relevant, as blockade of TRAIL by use of engineered oncolytic viruses enhanced the function of these viruses in extending survival in mouse models of GBM. Overall, our comprehensive investigation of GBM-associated astrocytes led to the discovery of a therapeutically targetable mechanism of immunosuppression operative in the TME of GBM, highlighting the need for further investigation into the immunological mechanisms performed by astrocytes in the context of GBM and other CNS diseases.

Provided herein are engineered herpes simplex viruses (oHSV) comprising a sequence encoding a tumor necrosis factor-related apoptosis-inducing ligand (TRAIL)-blocking monoclonal antibody or antigen-binding fragment thereof.

In some embodiments, the oHSV comprises one or more mutations that maximize infection and replication rates via fusogenic mutations.

In some embodiments, the oHSV comprises one or more microRNA target sites (miRTS) into the virus, e.g., selected from miR-124, miR-1, miR-143, miR-128, miR-219, miR-219a, miR-122, miR-204, miR-217, miR-137, miR-142, and miR-126.

In some embodiments, the oHSV comprises one or more mutations in UL37 that inhibit retrograde axonal transport.

In some embodiments, the oHSV comprises one or more deletions in the genes encoding viral ICP6 and ICP34.5 that reduce replication in non-replicating cells.

In some embodiments, the anti-TRAIL antibody is, or is an antigen binding fragment of, clone 75411, MAB375, RIK-2, Mapatumumab (HGS-ETR1), 4E7.24.3, TAB-160CQ, 4H6.17.8, 4E7.24.3, 43B353, HS10, HPAB-0460-WJ, HPAB-0412-Y, or 32A1380. In some embodiments, the antigen-binding fragment thereof is an scFv, scAb, Fab, or Fab₂.

In some embodiments, the oHSV comprises the oncolytic virus further comprises one or more additional transgenes. In some embodiments, the oHSV comprises the one or more additional transgenes comprise sequences encoding one, two, or more of interleukin-12 (IL-12), FLT3LG (extracellular domain), CCL4, and/or antagonists (optionally antibodies) to immune checkpoints PD-1 and CTLA-4.

Also provided herein are methods for the prevention (reduction of risk), treatment, and/or amelioration of a cancer in a subject. The methods comprise administering a therapeutically effective amount of an oncolytic virus as described herein, to a subject in need thereof. In some embodiments, the cancer is brain cancer, e.g., glioblastoma.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-J. Single-cell characterization of astrocytes in the glioblastoma TME.
(a) Schematic of study design to investigate astrocyte heterogeneity by scRNA-seq.
(b) UMAP plot of all cell types in human scRNA-seq data clustered by transcriptional similarity and divided by cell type.
(c) Cell type proportions by disease condition in human scRNA-seq data.
(d) UMAP plot of mouse astrocytes following unsupervised clustering by transcriptional similarities.
(e) Proportion of cells belonging to each cluster grouped by disease condition.
(f) UMAP plot of subclustered astrocytes analyzed from a published GBM scRNA-seq dataset [22].
(g) Cluster proportions of subclustered astrocytes.
(h) Feature plot showing expression of Tnfsf10 in mouse astrocyte clusters.
(i) Dot plot visualization of TNFSF10 expression level and percentage of expressing cells by condition.
(j) Dot plot visualization of TNFSF10 expression level and percentage of expressing cells by time to clinical GBM recurrence.

FIGS. 2A-E. A TRAIL-expressing astrocyte subset in the tumor microenvironment.
(a-b) Percentage and mean fluorescent intensity (MFI) of TRAIL-expressing TdTomato+ astrocytes in tumor-bearing mice 15 days after implantation (n=11 for each group). Representative histogram of surface TRAIL expression. Data shown as mean±SEM. ****p<0.0001, ***p<0.001, **p<0.01, *p<0.05, ns: p>0.05.
(c) Heatmap of differentially expressed genes in bulk RNA-seq of FACS-sorted TRAIL+ vs. TRAIL-astrocytes from tumor-bearing mice (n=4 per condition).
(d) Ingenuity Pathway Analysis (IPA) showing pathway enrichment of differentially expressed genes from bulk RNA-seq (p-val threshold 0.05).
(e) IPA analysis showing predicted upstream regulators responsible for the gene signature observed in TRAIL+ astrocytes compared to TRAIL-astrocytes.

FIGS. 3A-I. Astrocyte-mediated killing of T cells via TRAIL is a mechanism of immunosuppression in GBM.
(a) Schematic of lentivirus construct encoding targeting sgRNA sequence under the control of a U6 promoter, along with GFAP promoter-driven Cas9 gene and regulatory elements for stable transduction in vivo. Graph shows knock-down effect of TRAIL-lentivirus in TdTomato+ astrocytes isolated from naïve mice.
(b) Experiment design and survival curve for in vivo perturbation assay with astrocyte-specific knockdown of Tnfsf10, non-targeting Scrambled sgRNA or PBS sham-injected mice. Mice were monitored for 60 days and euthanized at humane endpoints based on weight.
(c) Percentage of Caspase-3/7+ CD4 or CD8 T cells isolated from the tumor site 15 days after implantation of GL261 tumors in mice receiving control- or Tnfsf10 in vivo genetic deletion.
(d) Percentage and absolute number of MHC I::GARC-1 neoantigen-specific CD8 T cells identified via tetramer staining.
(e) Schematic for in vitro co-culture system of astrocytes and T cells.
(f) Percentage of apoptotic CD4 or CD8 T cells following co-culture with TCM-stimulated astrocytes in vitro for 8 h.
(g) Percentage of Caspase-8+ CD4 or CD8 T cells following co-culture with TCM-stimulated astrocytes in vitro for 4 h.
(h-i) Effect of TRAIL blockade on CD4⁺ or CD8⁺ T-cell apoptosis induced by co-culture with TCM pre-treated astrocytes and determined by cleaved caspase-3/7 (h) or cleaved caspase-8 (i).

FIGS. 4A-K. Tumor-derived IL-11 regulates TRAIL expression via STAT3 signaling.
(a) Relative mRNA expression level of Tnfsf10 in GL261 TCM-stimulated astrocytes (left) or U87 TCM-stimulated astrocytes (right) relative to unstimulated astrocytes as measured by qPCR.
(b-c) Relative mRNA expression level of Tnfsf10 in TCM-stimulated astrocytes exposed to sub-lethal dose of 2 μM of the STAT3 inhibitor Niclosamide (BAY2353) or the STAT1 inhibitor Fludarabine, relative to vehicle-treated astrocytes.
(d) TNFSF10 mRNA level detected by bulk RNA-seq divided by phosphorylation level of STAT3 (at Y705) in GBM patients from a multi-omic study [24]. Only patients that ultimately succumbed to GBM were analyzed.
(e) Feature plot displaying expression of Il11ra1 in mouse astrocytes analyzed by scRNA-seq split by condition.
(f) Protein level of IL-11 in GL261 TCM supernatants. CT-2A cell line was included for comparison.
(g) Relative change in mRNA expression of Tnfsf10 in TCM-stimulated astrocytes upon neutralization of IL-11 via monoclonal antibodies, as compared to isotype antibody-treated TCM-stimulated astrocytes.
(h) Relative mRNA level of Tnfsf10 in astrocytes stimulated with recombinant murine IL-11 at 50 ng/ml compared to control medium.
(i) Relative mRNA level of Tnsf10 in rmIL-11 treated astrocytes upon STAT3 inhibition with BAY2353.
(j) Survival curve of GL261 glioma-bearing mice receiving astrocyte-specific in vivo genetic perturbation of Il11ra1.
(k) Survival analysis in TCGA data comparing patient subsets by IL-11 expression (1^{st} quartile vs. 4^{th} quartile).

FIGS. 5A-O. Therapeutic blockade of TRAIL extends survival by preventing T cell apoptosis.
(a) Schematic of oncolytic virus engineering strategy components.
(b) In vitro testing of our anti-TRAIL neutralizing antibody construct in astrocyte-T cell co-cultures stimulated with IFNg.
(c) Experimental design for therapeutic administration of oHSV 7 days post-tumor implantation.
(d) Survival curve of oHSV-treated mice receiving 3×10\hat{ }6 PFU intratumorally at day 7 post-implantation of GL261-mNectin1.
(e) Quantification of tumor burden by bioluminescence imaging of tumor-bearing mice before implantation and 9 days after treatment with oHSV.
(f) Percentage of apoptotic T cells in the TME of oHSV-treated mice at day 17 post-tumor implantation.
(g-i) HSV-1-(g) or GARC-1₇₇₋₈₅-specific CD8⁺ T cells (h), and activated GARC-1₇₇₋₈₅-specific PD-1⁺ CD8⁺ T cells (i) infiltrating the GL261 TME in HSV-1-treated mice 17 days post-implantation (n=8 per group).
(j,k) HSV-1 MHC-I tetramer⁺ and GARC-177-85 MHC-I tetramer⁺ activated/exhausted PD-1⁺ CD8⁺ T cells (j) and PD-1⁺ CD8⁺ T cells (k) in HSV-1-treated mice. Quantitative data shown in Extended Data FIG. 8b,d.
(l-o) IPA of bulk RNA-seq data analysis from FACS-sorted tumor-infiltrating CD8⁺ T cells (1), CD4⁺ T cells (m), microglia-derived (n) or monocyte-derived TAMs (o) in HSV-1-treated mice 17 days after tumor implantation (n=4 HSV-1^{Empty} except for monocytes, where n=3; n=5 for HSV-1^{α-TRAIL}); all pathways shown are P<0.05.

FIGS. 6A-I. FACS analysis of T-cell function in HSV-1-treated mice. (a) Apoptosis in T cells co-cultured with IFNγ-activated astrocytes and anti-TRAIL single-chain antibody, which was incorporated into HSV-1^{α-TRAIL} (n=5 anti-Isotype, n=3 anti-TRAIL). (b,c) Quantification of activated/exhausted tumor-infiltrating HSV-1 MHC-I tetramer⁺ PD-1⁺ CD8⁺ (b) or GARC-1₇₇₋₈₅ tetramer⁺ PD-1⁺ CD8⁺ (c) following HSV-1 treatment (n=8 per group). (d,e) Quantification of activated/exhausted tumor-infiltrating PD-1⁺ CD8⁺ (d) or PD-1⁺ CD4⁺ (e) T cells following HSV-1 treatment (n=8 per group). (f,g) FACS analysis of T-cell exhaustion markers in tumor-infiltrating CD8⁺ (f) or CD4⁺ (g) T cells with relevance for anti-tumor immunity in the context of TRAIL blockade (n=8 per group). (h,i) FACS analysis of cytokine expression in tumor-infiltrating CD8⁺ (h) or CD4⁺ (i) T cells (n=8 per group). Data shown as mean±SEM. Unpaired two-tailed t-test used.

FIGS. 7A-B. Graphical summary of findings. In this study, we describe the induction of TRAIL⁺ astrocytes in GBM. (a) TRAIL+ astrocytes are regulated by GBM-derived IL-11 via STAT3 signaling and limit anti-tumor immunity by inducing death receptor-mediated apoptosis in T cells. (b) Treatment with an oncolytic HSV engineered to express TRAIL blocking single-chain antibodies in the TME improves the anti-tumor response

### DETAILED DESCRIPTION

Described herein is targeted investigation of ‘bystander’ compartments in the TME that uncovered a subset of disease-relevant astrocytes and a therapeutically targetable interaction with tumor-directed T cells. In this study, we leveraged single-cell characterization of GBM-associated astrocytes and identified a TRAIL-expressing subpopulation associated with immunosuppression via T cell deletion. Further characterization of this subset revealed transcriptional regulation of TRAIL via STAT3 signaling. The pleiotropic cytokine IL11, produced by GBM cells, was found to induce this immunosuppressive astrocyte state, worsening survival in overexpression models, whereas perturbation of the IL11 signaling extended survival. Finally, therapeutic application of an engineered oncolytic virus expressing a TRAIL-blocking monoclonal antibody improved survival.

### Oncolytic Viruses

Described herein are engineered oncolytic viruses that express a TRAIL-blocking monoclonal antibody. Oncolytic herpes simplex viruses (oHSVs) are known in the art, see, e.g., WO/2017/132552; WO/2019/133847; WO/2022/150485; WO/2020/102285; WO/2020/142725; WO/2019/014623; WO/2019/023483; Koch et al., Cancers (Basel). 2020 Nov. 26; 12(12):3514; Glorioso et al., Curr Issues Mol Biol. 2021; 41:381-468. In some embodiments, provided are oncolytic herpes simplex virus-1 (oHSV) that include one or more mutations for maximizing infection and replication rates via fusogenic mutations (see, e.g., Cristi et al., Front Mol Biosci. 2022; 9:831091; Thomas et al., J Immunother Cancer. 2019 Aug. 10; 7 (1): 214). To ensure safety and tolerability of this treatment, a microRNA attenuation mechanism can also be incorporated, e.g., incorporation of microRNA target sites (miRTS) into the virus (see, e.g., Leber et al., Mol Ther Oncolytics. 2018 Jun. 29; 9:30-40; Kennedy et al., Mol Ther Oncolytics. 2020 Aug. 8; 18:476-490). In some embodiments, the one or more miRNAs are selected from miR-124, miR-1, miR-143, miR-128, miR-219, miR-219a, miR-122, miR-204, miR-217, miR-137, miR-142, and miR-126 (see, e.g., WO2019023483 and WO2022150485).

Additional safety mechanisms that can be incorporated include mutations in UL37 to inhibit retrograde axonal transport (Kennedy et al., Mol Ther Oncolytics. 2020 Aug. 8; 18:476-490); retargeting the HSV tropism to cancer-specific receptors (Campadelli-Fiume et al., Viruses. 2016 March; 8 (3): 63); and/or deletions in the genes encoding viral ICP6 and ICP34.5 (Nakashima et al., Clin Cancer Res. 2018 Jun. 1; 24 (11): 2574-2584), so as to protect non-malignant cells in the brain from viral infection and lysis. In some embodiments, the oHSV includes one or more mutations described in HF10 (Ushijima et al., Microbes Infect. 2007 February; 9(2): 142-9).

In some embodiments, the oHSV is a oNCR-142 as described in WO2019023483, or another virus described therein, e.g., in Table 18 thereof, reproduced here as Table A:

TABLE A
(from WO2019023483): HSV constructs comprising candidate miR-TS cassettes

| Construct | ICP27 | UL8 | ICP34.5 | ICP4 | UL42 |
|---|---|---|---|---|---|
| ONCR-142 | 219a-5p (4x) | 137-3p (4x) | 128-3p^{M} (4x) | 124-3p (4x) | X |
| ONCR-154 | 122-5p (4x) | 208b-3p (4x) | 204-5p (4x)^{ψ} | 1-3p (4x) | |
| | 128-3p (4x) | 126-3p (4x) | 219a-5p (4x)^{ψ} | 143-3p (4x) | |
| ONCR-156 | 219a-5p (4x) | 137-3p (4x) | | 124-3p (4x) | X |
| | 122-5p (4x) | 208b-3p (4x) | | 1-3p (4x) | |
| | 128-3p (4x) | 126-3p (4x) | | 143-3p (4x) | |
| ONCR-158 | 219a-5p (4x) | 137-3p (4x) | 128-3p^{M} (4x) | 124-3p (4x) | |
| | 122-5p (4x) | 208b-3p (4x) | 204-5p (4x) | 1-3p (4x) | |
| | 128-3p (4x) | 126-3p (4x) | 219a-5p (4x) | 143-3p (4x) | |
| ONCR-157 | 219a-5p (4x) | 137-3p (4x) | 128-3p^{M} (4x) | 124-3p (4x) | X |
| | 122-5p (4x) | 217-5p (4x) | 204-5p (4x) | 1-3p (4x) | |
| | 128-3p (4x) | 126-3p (4x) | 219a-5p (4x) | 143-3p (4x) | |
| ONCR-159 | 219a-5p (4x) | 137-3p (4x) | 128-3p^{M} (4x) | 124-3p (4x) | X |
| ONCR-165 | 122-5p (4x) | 217-5p (4x) | 204-5p (4x) | 1-3p (4x) | |
| ONCR-166 | 128-3p (4x) | 126-3p^{M} (4x) | 219a-5p (4x) | 143-3p (4x) | |
| ONCR-167 | | | | | |
| ONCR-168 | | | | | |
| ONCR-169 | | | | | |
| ONCR-170 | | | | | |
| ONCR-171 | | | | | |
| ONCR-172 | | | | | |
| ONCR-173 | | | | | |
| ONCR-174 | | | | | |
| ONCR-175 | | | | | |
| ONCR-176 | | | | | |
| ONCR-177 | | | | | |
| ONCR-160 | 219a-5p (4x) | 137-3p (4x) | 128-3p^{M} | 124-3p (4x) | X |
| | 122-5p (4x) | 208b-3p (4x) | 204-5p (4x)^{ψ} | 1-3p (4x) | |
| | 128-3p (4x) | 126-3p (4x) | 219a-5p (4x)^{ψ} | 143-3p (4x) | |
| ONCR-161 | 219a-5p (4x) | 137-3p (4x) | 128-3p^{M} (4x) | 124-3p (4x) | X |
| | 122-5p (4x) | 208b-3p (4x) | 204-5p (4x)^{ψ} | 1-3p (4x) | |
| | 128-3p (4x) | 127 (4x) | 219-5p (4x)^{ψ} | 143-3p (4x) | |
| ONCR-162 | 219a-5p (4x) | 137-3p (4x) | 128-3p^{M} (4x) | 124-3p (4x) | X |
| | 122-5p (4x) | 208b-3p (4x) | 204-5p (4x)^{ψ} | 1-3p (4x) | |
| | 128-3p (4x) | 128-3p (4x) | 219-5p (4x)^{ψ} | 143-3p (4x) | |
| ONCR-163 | 219a-5p (4x) | 137-3p (4x) | 128-3p^{M} (4x) | 124-3p (4x) | X |
| | 122-5p (4x) | 208b-3p (4x) | 204-5p (4x)^{ψ} | 1-3p (4x) | |
| | 128-3p (4x) | 129 (4x) | 219-5p (4x)^{ψ} | 143-3p (4x) | |
| ONCR-164 | 219a-5p (4x) | 137-3p (4x) | 128-3p^{M} (4x) | 124-3p (4x) | X |
| | 122-5p (4x) | 208b-3p (4x) | 204-5p (4x)^{ψ} | 1-3p (4x) | |
| | 128-3p (4x) | 130 (4x) | 219-5p (4x)^{ψ} | 143-3p (4x) | |

^{ψ}one of the 4 target sequences was non-functional due to cloning error
^{M}comprises a modified target sequence

In order to disrupt the T cell-deletive function of tumor necrosis factor (TNF)-related apoptosis-inducing ligand (TRAIL)-expressing astrocytes in a therapeutic manner, a transgene construct encoding a TRAIL-blocking monoclonal antibody can be included (oHSV^{α-TRAIL}). The term “antibody” as used herein refers to an immunoglobulin molecule or an antigen-binding portion thereof. Examples of antigen-binding portions of immunoglobulin molecules include scFv, scAb, F(ab), and F(ab′)₂ fragments, which retain the ability to bind antigen and the blocking ability of the full antibody. The antibody can be chimeric, de-immunized or humanized, fully human, non-human, (e.g., murine), or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. Methods for making antibodies and fragments thereof are known in the art, see, e.g., Harlow et. al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec. 13, 2006); Kontermann and Dübel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dübel, Handbook of Therapeutic Antibodies: Technologies, Emerging Developments and Approved Therapeutics, (Wiley-VCH; 1 edition Sep. 7, 2010).

TRAIL-blocking antibodies, and antigen-binding fragments thereof that block TRAIL signaling, are known in the art; and are commercially available, including antibody clones 75411 (Sigma Aldrich), MAB375 (R&D Systems), and RIK-2, Mapatumumab (HGS-ETR1), 4E7.24.3, TAB-160CQ, 4H6.17.8, 4E7.24.3, 43B353, HS10, HPAB-0460-WJ, HPAB-0412-Y, and 32A1380 (eBioscience). In some embodiments, the VH and VL regions comprise the following: Sequence of the VH region:

(SEQ ID NO: 1)
QVTLKESGPGILQPSQTLSLTCTFSGFSLTTHGMGVGWIRQPAGKGLEW
LANIWWDDDKFHSPSLKNRLTISKDTSNNQAFLRITNVDTADAAIYYCA
RISWERAYYFDYWGQGVMVTVSSAETTAPSVYPLAPGTALKSNSMVTLG
CLVKGYFPEPVTVTWNSGALSSGVHTFPAVLQSGLYTLTSSVTVPSSTW
SSQAVTCNVAHPASSTKVDKKIVPRECNPCGCTGSEVSSVFIFPPKTKD
VLTITLTPKVTCVVVDISQNDPEVRFSWFIDDVEVHTAQTHAPEKQSNS
TLRSVSELPIVHRDWLNGKTFKCKVNSGAFPAPIEKSISKPEGTPRGPQ
VYTMAPPKEEMTQSQVSITCMVKGFYPPDIYTEWKMNGQPQENYKNTPP
TMDTDGSYFLYSKLNVKKETWQQGNTFTCSVLHEGLHNHHTEKSLSHSP
G;

sequence of the VL region:

(SEQ ID NO: 2)
DVQMTQSPSFLSASVGERVTLSCRASQNVDTYLDWYQQKLGEAPKLLIY
NTNNLHTGIPSRFSGSGSGTDYTLTISSLQPEDVATYFCLQRNRWLTFG
SGTKLEIKRADAAPTVSIFPPSMEQLTSGGATVVCFVNNFYPRDISVKW
KIDGSEQRDGVLDSVTDQDSKDSTYSMSSTLSLTKVEYERHNLYTCEVV
HKTSSSPVVKSFNRNEC.

Alternatively or in addition, a transgene construct encoding an IFNγ-blocking monoclonal antibody, or an antigen-binding fragment thereof that blocks IFNY signaling, can be included. IFNγ-blocking monoclonal antibodies are known in the art, and are commercially available, including antibody clones MD-1 and NIB42 (both available from eBioscience), HuZAF (Fontolizumab, Facet Biotech; Creative Biolabs), and clones CBL766, CBL813, 43.B3, 7H58, CBL201, PABX-112, PABX-112-S, PABX-112-F, TAB-423CQ, AF2, P2C11, D9D10, B-B1, and B133.5 (Creative Biolabs).

Also provided herein are pharmaceutical compositions comprising the oncogenic viruses described herein, and a physiologically acceptable carrier. Suitable compositions include those described in WO2019023483 and WO2022150485.

In some embodiments, the oncolytic virus further comprises one or more additional transgenes; alternatively, the methods can include administering a combination of oncolytic viruses comprising a plurality of transgenes. The additional transgenes can include, e.g., sequences encoding one, two, or more of interleukin-12 (IL-12), FLT3LG (extracellular domain), CCL4, and antagonists to immune checkpoints PD-1 and CTLA-4 (see, e.g., Haines et al., Cancer Immunol Res. 2021 March; 9 (3): 291-308; Leoni et al., PLOS Pathog. 2018 August; 14 (8): e1007209); GADD34 (Nakashima et al., Clin Cancer Res. 2018 Jun. 1; 24 (11): 2574-2584).

Also provided herein are methods for the prevention (reduction of risk), treatment, and/or amelioration of a cancer in a subject. In some embodiments, the methods described herein comprise administering an effective amount (e.g., a therapeutically effective amount) of an oncolytic virus described herein to a subject in need thereof, wherein the virus expresses a TRAIL-blocking monoclonal antibody or antigen-binding portion thereof that retains blocking ability. In some embodiments, the cancer is brain cancer, e.g., glioblastoma.

The compositions can be introduced by catheter (see U.S. Pat. No. 5,328,470) or by stereotactic injection, e.g., optionally into a tumor, or into the cisterna magna, cerebral ventricles, or lumbar intrathecal space (e.g., Chen et al., PNAS USA 91:3054-3057 (1994)). In some embodiments, delivery methods of the viruses described herein can include intravenous, intrathecal, intracerebroventricular, intracisternal, intranerve, intra-articular, intramuscular, subcutaneous, intradermal, epidural, intracisternal, intracerebroventricular, transforaminal, selective nerve root, and stereotactic intraparenchymal administration in the dorsal root or trigeminal ganglia or spinal cord.

### EXAMPLES

The invention is further described in the following examples, which do not limit the scope of the invention described in the claims.

### Materials and Methods

The following materials and methods were used in the Examples below.

Mice. C57BL/6J mice (The Jackson Laboratory, 000664) were used as a genetic background for all in vivo experiments. Mice were housed in a conventional, pathogen-free facility at the Hale Building for Transformative Medicine under a standard 12 h light/dark cycle at 20-23° C. and 50% humidity with access to water and food ad libitum, in accordance with guidelines for care and experimentation defined by the Institutional Animal Care and Use Committee (IACUC). Transgenic mouse genotypes were confirmed by PCR (Transnetyx). When Aldh111^{Cre-ERT2/TdTomato} astrocyte reporter mice (The Jackson Laboratory, #031008 and #007909) were used, 200 mg/kg Tamoxifen (Sigma-Aldrich, T5648) diluted in corn oil (Sigma-Aldrich, C8267) was administered intraperitoneally 2-4 weeks before starting the experiment and TdTomato induction was confirmed by flow cytometry at the time of measurement.

Human GBM samples. Resected human GBM and healthy brain tissue samples were obtained from the Department of Oncologic Pathology at Dana Farber Cancer Institute and stored at −80° C. De-identified clinical data, including patient age, sex, diagnosis, treatment history, isocitrate dehydrogenase (IDH) mutation status, O⁶-methylguanine-DNA methyltransferase (MGMT) promoter methylation status and cytogenetic report of copy number variations (CNV) is included in Table 1.

TABLE 1
GBM sample characteristics.

| | | | | | MGMT | | |
|---|---|---|---|---|---|---|---|
| | | | | IDH | promoter | Relevant genetic | Previous |
| Age | Sex | Diagnosis | Timepoint | status | methylation | alterations | therapy |
| 65 | M | GBM | New | Unknown | Partially | EGFR amplification | — |
| | | | Diagnosis | | methylated | PTEN loss | |
| | | | | | | 1-2 copy | |
| | | | | | | CDKN2A/B loss | |
| | | | | | | Polysomy 7 | |
| | | | | | | Monosomy 10 | |
| 54 | M | GBM | New | Mutant | Methylated | RB1 loss | — |
| | | | Diagnosis | | | PTEN loss | |
| 19 | F | GBM | New | Mutant | Methylated | AKT3 gain | — |
| | | | Diagnosis | | | PIK3CA gain | |
| | | | | | | PDGFRA | |
| | | | | | | amplification | |
| | | | | | | MYC gain | |
| | | | | | | Homozygous | |
| | | | | | | CDKN2A/B loss | |
| | | | | | | PTEN loss | |
| | | | | | | 10q loss | |
| 86 | M | GBM | New | WT | Absent | SOX2 amplification | — |
| | | | Diagnosis | | | PDGFRA | |
| | | | | | | amplification | |
| | | | | | | PTEN loss | |
| | | | | | | CDKN2A/B loss | |
| | | | | | | Polysomy 7 | |
| | | | | | | Monosomy 10 | |
| 28 | F | GBM | New | Mutant | Methylated | PDGFRA | — |
| | | | Diagnosis | | | amplification | |
| | | | | | | CCND2 gain | |
| | | | | | | CDK4 amplification | |
| | | | | | | RB1 loss | |
| | | | | | | Heterozygous 7q | |
| | | | | | | gain | |
| | | | | | | Heterozygous 10q | |
| | | | | | | loss | |
| | | | | | | Monosomy 13 | |
| | | | | | | Monosomy 19 | |
| 74 | M | GBM | New | WT | Methylated | EGFR amplification | — |
| | | | Diagnosis | | | PTEN loss | |
| | | | | | | CDKN2A/B loss | |
| | | | | | | CCND2 | |
| | | | | | | amplification | |
| | | | | | | Polysomy 7 | |
| | | | | | | Monosomy 10 | |
| | | | | | | Monosomy 22 | |
| 64 | M | GBM | Recurrent | WT | Methylated | PTEN loss | Temodar |
| | | | | | | 1-2 copy | |
| | | | | | | CDKN2A/B loss | |
| | | | | | | RB1 loss | |
| | | | | | | 6q loss | |
| | | | | | | Polysomy 7 | |
| | | | | | | Monosomy 10 | |
| | | | | | | Monosomy 13 | |
| 58 | F | GBM | New | WT | Methylated | MDM4 gain | — |
| | | | Diagnosis | | | EGFR amplification | |
| | | | | | | PTEN loss | |
| | | | | | | Heterozygous | |
| | | | | | | CDK6 gain | |
| | | | | | | 1-2 copy | |
| | | | | | | CDKN2A/B los | |
| | | | | | | CCND2 gain | |
| | | | | | | Monosomy 10 | |
| 75 | F | GBM | New | WT | Partially | lp deletion, | — |
| | | | Diagnosis | | methylated | CDKN2C loss | |
| | | | | | | Polysomy 7 | |
| | | | | | | Monosomy 10 | |
| | | | | | | Monosomy 13 | |
| | | | | | | Monosomy 17 | |
| | | | | | | Monosomy 22 | |
| 67 | M | GBM | Recurrent | WT | Methylated | SOX2 gain | Temodar |
| | | | | | | TERT gain | |
| | | | | | | QKI gain | |
| | | | | | | EGFR amplification | |
| | | | | | | PTEN loss | |
| | | | | | | Homozygous | |
| | | | | | | CDKN2A/B loss | |
| | | | | | | Polysomy 7 | |
| | | | | | | Monosomy 10 | |
| 60 | F | GBM- | Autopsy | Unknown | Methylated | | — |
| | | Control | | | | | |
| | | tissue | | | | | |
| 51 | M | GBM- | Autopsy | Unknown | Absent | | — |
| | | Control | | | | | |
| | | tissue | | | | | |
| 58 | M | GBM- | Autopsy | Unknown | Absent | | — |
| | | Control | | | | | |
| | | tissue | | | | | |

snRNA-seq. To avoid transcriptional artifacts resulting from bias in recovery of different cell types from frozen tissue, a previously described dissociation protocol for nuclei isolation was adapted [35]. Briefly, frozen tissue was minced with a razor blade and mechanically homogenized in Nuclei EZ Lysis Buffer (Sigma-Aldrich). After incubation for 5 min, the homogenate was filtered through a 70 μm strainer mesh. Two washing steps were performed with a 1% solution of bovine serum albumin (BSA) (Sigma-Aldrich, A3294-100G) in phosphate buffered saline (PBS) (Thermo Fisher Scientific, 14190250), with addition of 0.2 U/μL of RNase Inhibitor (Lucigen, 30281-2). Centrifugation occurred at 500×g for 5 minutes at 4° C. To separate nuclei from cell debris, nuclei suspensions were incubated for 10 min with 10 μg/mL 4′,6-Diamidin-2-phenylindol (DAPI) (Sigma, #D9542) in wash buffer to stain DNA. Subsequently, DAPI⁺ nuclei were sorted via fluorescence activated cell sorting (FACS) and utilized for Drop-Seq as described below.

Drop-Seq. Drop-Seq was used for scRNA-seq via co-encapsulation of uniquely barcoded beads and single cells in nanoliter droplets prior to cell/nucleus lysis and transcriptome capture. Following the protocol developed by Macosko et al. [36], barcoded MACOSKO-2011-10 B beads (Chemgenes) linked to 10⁸ uniquely barcoded oligonucleotides (5′-Bead-Linker-TTTTTTTAAGCAGTGGTATCAACGCAGAGTACJJJJJJJJJJJJNNNNNNNNTTTT TTTTTTTTTTTTTTTTTTTTTTTTTT-3′; SEQ ID NO:3) were resuspended in a wash solution made of 10 mM Tris pH 8.0 (Sigma-Aldrich, T3038), 1 mM EDTA (Amresco, E177-100ML) and 0.01% Tween-20 (Sigma-Aldrich, P9416) (TE-TW) at a density of 3×10⁵/mL and stored at 4° C. Immediately prior to use, beads were centrifuged at 1000×g and resuspended in 1 mL of lysis buffer composed of 570 μL OptiPrep (Sigma-Aldrich, D1556-250ML), 120 μL 20% Ficoll PM-400 (Cytiva, 17-0300-10), 20 μL 10% Sarkosyl (Sigma-Aldrich, L7414-50ML), 40 μL 0.5M EDTA (Amresco, E177-100ML), 200 μL 1M Trizma hydrochloride solution pH 8.0 (Sigma-Aldrich, T2663-1L), 50 μL 1M dithiothreitol (Thermo Fisher Scientific, P2325) (DTT). Sorted cells or nuclei were taken up in 16% OptiPrep at a concentration of 2.5×10⁵/mL in 1 mL. If fewer cells or nuclei were available, Fluorinert FC-40 (Sigma Aldrich, F9755-250ML) was used as a backflow liquid. If fewer than 1×10⁵ cells were available, bead concentrations were doubled to 6×10⁵/mL to increase the capture rate, which follows a Poisson distribution. Beads and cells were taken up in a 3 mL syringe (BD), adding a magnetic disk (VP Scientific, 772DP-N42-5-2) and using a magnetic stirrer (VP Scientific) to homogenize bead suspensions. QX200 Droplet Generation Oil for EvaGreen (BioRad, 1864006) was taken up in a 10 ml syringe (BD). Three Legato 100 syringe pumps (KD Scientific) were loaded with oil, cell, and bead syringes, and PE/2 polyethylene microtubing (Scientific Commodities Inc., BB31695-PE/2) was used to connect them to the device input holes of PDMS devices placed on an inverted light microscope with an attached Fire-i 830b camera (Unibrain). Fire-i (Unibrain) was used to visualize rapidly flowing droplets with lowest shutter speed and highest exposure and gain settings. The device was initially primed with oil, prior to connecting cell and lastly bead flows at flow rates of 4500, 1500 and 1500 μL/h, respectively. Output flow containing droplets was collected for 20-25 min or until the cell suspension was used up. For droplet breakage, approximately ⅓ of excess oil volume was added as 97% 1H,1H,2H,2H-Perfluoro-1-octanol (Sigma-Aldrich, 370533), along with 30 mL of 6× sodium chloride-sodium citrate (SSC) buffer (National Diagnostics, EC-873). Emulsions were shaken a few times and centrifuged at 1000×g for Imin, resulting in a visible bead layer floating at the interface of oil and SSC. In the following steps, as much of the oil and SSC as possible was removed, while topping up with fresh SSC and centrifuging to wash the beads prior to reverse transcription (RT) into cDNA. Beads were washed in 300 μL of 1×RT buffer (Thermo Fisher Scientific, EP0742) prior to incubation in 200 μL RT mix composed of 75 μL H2O, 40 μL 5×RT buffer, 40 μL 20% Ficoll PM-400 (Cytiva, 17-0300-10), 20 μL 1 mM dNTPs (Thermo Fisher Scientific, R0194), 5 μL RNase Inhibitor (Lucigen, 30281-2), 10 μL 50 μM Template Switch Oligo (AAGCAGTGGTATCAACGCAGAGTGAATrGrGrG (SEQ ID NO:4), IDT) and 10 μL (with 2000U) Maxima H Minus Reverse Transcriptase (Thermo Fisher Scientific, EP0742) for 30 min at room temperature and 90 min at 42° C. (with rotation in both cases). Reaction mixtures were then washed with 1 mL of a solution containing 10 mM Tris pH 8.0, 1 mM EDTA and 0.5% sodium dodecyl sulfate (Sigma-Aldrich, L3771-100G) (TE-SDS), twice with 1 mL of TE-TW, and once with 1 mL 10 mM Tris pH 8.0. To remove bead-linked oligonucleotides that had not captured an mRNA molecule, beads were incubated in a mix of 20 μL 10× Exonuclease I buffer (Thermo Fisher Scientific, EN0581), 170 μL H2O and 10 μL (with 200U) Exonuclease I (Thermo Fisher Scientific, EN0581) for 45 min at 37° C. with rotation. After washing once with TE-SDS, twice with TE-TW and once with 1 mL H2O beads were counted using a hemocytometer chamber and divided into 4000 beads per aliquot in PCR tubes. A PCR mix containing 24.6 μL H2O, 0.4 μL 100 μM SMART PCR primer (AAGCAGTGGTATCAACGCAGAGT (SEQ ID NO:5)) (IDT) and 25 μL 2× Kapa HiFi Hotstart Readymix (Kapa Biosystems, 7958927001) were added. PCR was performed in a thermocycler with the following program to amplify cDNA molecules: 95° C. (3 min); 4 cycles of: 98° C. (20 s), 65° C. (45 s), 72° C. (3 min); 9 cycles of: 98° C. (20 s), 67° C. (20 s), 72° C. (3 min); 72° C. (5 min); 4° C. hold. PCR products were purified using AMPure XP beads (Beckman Coulter, A63881) at a 0.6× ratio according to manufacturer's protocol, eluting all samples in 50 μL molecular grade H2O (Thermo Fisher Scientific, AM9937). At this point, the distribution of fragment sizes within cDNA libraries was quantified using a 2100 Bioanalyzer (Agilent) instrument with High Sensitivity DNA Kit (Agilent, 5067-4626). Library concentration and quality was assessed by a rounded curve with average sizes around 700 bp. In cases where DNA concentration was not sufficient, additional PCR cycles with the following conditions were added as needed: 98° C. (20 s), 67° C. (20 s), 72° C. (3 min). 600 pg of cDNA library in 5 μL were used for fragmentation and addition of sample tags by means of the Nextera XT DNA Library Prep Kit (Illumina, FC-131-1096). cDNA was added to 10 μL of Nextera Tagment DNA buffer and 5 μL of Amplicon Tagment Mix. After incubation for 5 min at 55° C., 5 μL of Neutralize Tagment buffer was added. After 5 min at room temperature, a mix containing 15 μL Nextera PCR Mix, 8 μL H2O, 1 μL 10 μM New-P5-SMART PCR hybrid oligo (AATGATACGGCGACCACCGAGATCTACACGCCTGTCCGCGGAAGCAGTG GTATCAACGCAGAGT*A*C (SEQ ID NO:6)) (IDT), and 1 μL of a 10 μM sample-specific Nextera indexing oligonucleotide (Illumina, FC-121-1011). Samples underwent PCR with the following program: 95° C. (30 s); 12 cycles of: 95° C. (10 s), 55° C. (30 s), 72° C. (30 s); 72° C. (5 min); 4° C. hold. cDNA was purified AMPure XP beads at a 0.6× ratio and eluted in 10 μL H2O. Library distribution was analyzed using a 2100 Bioanalyzer and the molarity-weighted average fragment size was calculated. The Library Quantification Kit (Kapa Biosystems) was used to determine library concentrations by qPCR according to standard protocol. Finally, 4 μL samples were pooled at an equimolar ratio of 5 nM. Finally, samples were sequenced using NovaSeq S2 at with a Custom Read 1 primer (GCCTGTCCGCGGAAGCAGTGGTATCAACGCAGAGTAC (SEQ ID NO:7)), estimating around 25,000 reads per STAMP. STAMPs were estimated by multiplying the cell number used (determined by flow rate, run time and concentration) with the co-encapsulation probability (given by the product of the encapsulation probability of either cells or beads). The co-encapsulation probability of single units follows a Poisson distribution as a function of droplet volume and cell or bead concentration and was 2.5% for mouse samples and on average 1.4% for human nuclei.

PDMS device fabrication. Microfluidic devices were microfabricated by regular photolithography techniques at the Harvard Medical School Microfluidics/Microfabrication Core Facility. To generate a master mold with features of 125 μm height, a silicon wafer (University Wafer) was spin-coated with the epoxy-based negative photoresist SU-8 (Kayaku Advanced Materials) and using a Mask Aligner exposed to ultraviolet light through a photomask (CAD/Art) containing the Drop-seq pattern (mccarrolllab.org/dropseq). Spin coating and exposure conditions were determined following the SU-8 manufacturer's protocol. The photoresist was then treated with SU8 developer and isopropanol was used to rinse off undeveloped SU-8. Finally, the wafer was heated at 150° C. for 5 min. For replica molding with polydimethylsiloxane (PDMS), SYLGARD 184 silicone elastomer and DOWSIL 184 curing agent (Dow, DC4019862) were mixed at a 1:10 ratio. The mixture was poured into the master mold and air bubbles removed by placing in a vacuum degassing chamber for 2-3 hours. Finally, the elastomer was cured at 65° C. for at least 4 h or preferably overnight. 0.75 mm wide holes were punched using a biopsy punch (Rapid Core). Devices were bonded to glass microslides (Fisher Scientific) by treatment with oxygen plasma (Technics Plasma Etcher 500-II) and baking at 150° C. for 10 min. Finally, channels were rendered hydrophobic by treatment with filter-sterilized Aquapel (Aquapel Glass Treatment) for 5 min. Aquapel was removed with pressurized oxygen and channels washed with Fluorinert FC-40 (Sigma-Aldrich, F9755-250ML) and again flushed with pressurized air. Finally, PDMS devices were baked at 65° C. overnight and sealed with Scotch Tape until use.

Orthotopic GL261 model and delivery of lentivirus. 1×10⁵ GL261-Luc2 cells (ATCC) were implanted intracranially. GL261 cells with passage number <10 were detached using Accutase (Stemcell Technologies) and cell suspensions prepared in serum-free DMEM. The surgical procedure was performed following IACUC guidelines for survival surgery. Briefly, a small animal stereotaxic frame (David Kopf Instruments) was placed within a laminar flow cabinet and a sterile field containing all surgical instruments and tools was established. An activated charcoal filter (VetEquip) was connected to the outflow to adsorb Isoflurane. Mice were individually placed in an anesthesia induction chamber and a flow of Isoflurane (Patterson Veterinary) at 2.5-3 L/h mixed with oxygen at 2.5 L/h was initiated. The top of the skull was shaved and disinfected using isopropyl swabs (Becton Dickinson) and application of the antiseptic Betadine (Purdue Products). For intracranial injection, the mouse skull was fixed on the stereotaxic frame using ear bars and a mouthpiece for incisor fixation and gas supplementation. Sufficient depth of anesthesia was confirmed by a toe pinch, and mouse vital signs were monitored throughout the procedure. Following a 2 cm incision on the scalp, the injection coordinates were determined from bregma. A hole was drilled at the +1.8 mm (lateral) and +0.5 mm (anterior) position. A 25 μl glass syringe (Hamilton) was inserted until-3.5 mm depth and slowly retracted 0.5 mm to the final injection site at −3 mm depth. 3 μL of GL261 cell suspension were gradually injected at a rate of 1 μl/min. Control mice received a sham injection of 3 μL DMEM. For lentivirus administration, 10 μl containing 10⁷ IU of lentivirus were injected at a rate of 2 μl/min at the same location of the tumor implant. After an additional minute, the needle was slowly retracted so as to avoid liquid backflow. The skin was sutured using Ethilon 4-0 non-absorbable filaments (Ethicon) in a simple interrupted pattern. Mice received one perioperative subcutaneous injection of the slow-release analgesic Buprenorphine-SR at a dose of 1 mg/kg body weight. Mouse weight was monitored daily, along with levels of activity and signs of wound inflammation or pain. For assessment of tumor burden, in vivo bioluminescence imaging was used. Briefly, mice were anesthetized in a gas chamber as described above, and 0.15 mg/kg D-Luciferin (Cayman Chemical) in PBS was injected intraperitoneally. After 10 min, luminescence at 560 nm wavelength was captured multiple times at high sensitivity and 30s exposure time inside an In-Vivo Xtreme Optical/X-ray imaging system (Bruker). Bruker MI SE v7 (Bruker) software was used for quantitation of photons per second (P/s) within the area of the mouse skull, using the highest intensity recording to capture the peak of bioluminescence. X-ray image overlays were used to limit ROI to mouse skull area. To account for background noise, the median value on the region's perimeter was subtracted from total P/s, resulting in net P/S as a final measure of tumor burden.

Cell culture. The GL261-Luc2 cell line (Caliper Life Sciences) was cultured in high-glucose DMEM (Gibco, 11965118) supplemented with 10% fetal bovine serum (FBS) (Gibco, 10-438-026), 1% penicillin/streptomycin (Thermo Fisher Scientific, 15140122) and 100 ug/mL G418 Geneticin (Thermo Fisher Scientific, 10131027). U-87 MG cells (ATCC, HTB-14) were cultured in the same medium as GL261-Luc2 cells, except no selection antibiotic G418 was added. HEK293FT cell line (Invitrogen, R70007) was cultured with DMEM/F12+GlutaMAX (Thermo Fisher Scientific, 10565-042) with 10% FBS, 1% penicillin/streptomycin and 100 ug/mL G418 Geneticin (Thermo Fisher Scientific, 10131027). Hep G2 cells (ATCC, HB-8065) were cultured in high-glucose DMEM supplemented with 10% FBS and 1% penicillin/streptomycin. GL261 TCM was prepared as previously described by culturing 6×10⁶ cells in 10 mL selection antibiotic-free culture medium for 24 h. Culture supernatants were then centrifuged and sterile filtered using a 22 μm filter to remove cellular debris, and aliquots stored at −80° C. until use.

Generation of GL261 cell lines. Briefly, 1.5×10⁵ cells were seeded in 6-well plates and transfected by dropwise addition of DNA-lipid complexes made with 2.5 ug DNA at a DNA to Lipofectamine 3000 (Thermo Fisher Scientific, L3000008) ratio of 1:3 in Opti-MEM reduced serum medium+GlutaMAX (Thermo Fisher Scientific, 51985034). 48 h post-transfection, antibiotic selection was carried out with 800 g/mL Zeocin (Invivogen, ant-zn-1) for 2 weeks. Successfully transfected, antibiotic-resistant clones were further expanded and maintained at 300 ug/ml Zeocin thereafter. For IL-11 overexpression, parental GL261-Luc cells were transfected with a cDNA construct encoding mouse IL-11 and GFP (Sino Biological, MG50117-ACG) as described above, and selected with Hygromycin B (Cayman Chemical Company, 14291) at 500 ug/ml. Next, polyclonal populations were FACS sorted to purify highly GFP positive cells and maintained in 150 ug/ml Hygromycin B. Finally, growth dynamics were calculated by seeding 20,000 cells and performing cell counting via flow cytometry after 2, 3, 4 or 7 days. IL-11 expression was screened by qPCR and by ELISA. Clone 2 was selected based on exhibiting unchanged growth and successfully overexpressing IL-11. GL261 cells expressing mNectin-1 (GL261-mNectin1) were generated by lentiviral transduction of mNectin-1 cDNA followed by selection with 2 μg/mL puromycin (Gibco, A1113803). Single cells were obtained by limiting dilution and clones were screened for mNectin-1 expression by qPCR (Nectin1, Mm00445392_m1).

Isolation of mouse CNS cells. For cell isolation from fresh mouse brain tissue, anesthetized mice were perfused with 20 mL cold PBS. The brain was minced and enzymatically dissociated for 20 min, 37° C. and 100 rpm shaking in a solution containing 0.66 mg/mL Papain (Sigma-Aldrich, #P4762) in HBSS w/o Mg/Ca (Thermo Fisher Scientific, 14175095), followed by a second incubation at the same conditions with equal volume of DMEM supplemented with Collagenase D (Roche, #11088858001) and DNase I (Thermo Fisher Scientific, #90083) in the concentration of 0.66 mg/mL and 8 U/mL respectively. Using a 10 mL serological pipette, the suspension was dissociated and filtered through a 70 μm cell strainer (Fisher Scientific, #22363548) into a 50 mL tube. After centrifugation at 600×g for 5 min, the pellet was resuspended in 10 mL of a solution containing 30% Percoll (GE Healthcare Biosciences) in PBS. Following gradient centrifugation for 25 min at 600×g with lowest acceleration and deceleration speeds, the layer of myelin accumulating at the top was aspirated. The cell pellet was washed with PBS and used for downstream applications.

Isolation of mouse splenocytes. Mouse spleens were dissected out of anesthetized mice prior to cardiac perfusion, mechanically mashed through 70 μm nylon strainers (Thermo Fisher Scientific, 22-363-548) by use of a syringe plunger, and washed with HBSS w/o Mg/Ca (Thermo Fisher Scientific, 14175095). Next, spleen tissue was incubated in ImL ACK Lysis Buffer (Gibco, A10492) for 5 min at 4° C. to lyse red blood cells, washed in HBSS, centrifuged at 500×g and resuspended in PBS with 0.5% BSA (Sigma-Aldrich, A3294-100G) and 2 mM EDTA (VWR, E177-100ML) for downstream applications.

Lentivirus production for in vivo CRISPR-Cas9 deletion. We have previously described lentiviral backbones used perform GFAP promoter-driven

genetic deletions in vivo [12], which are derived from lentiCRISPRv2 (Feng Zhang, Addgene plasmid #52961) and express Cas9 driven by the ABC1D gfa2 GFAP promoter [38], while sgRNA is under U6 promoter control. In order to exchange 20 nt-long sgRNA sequences to target different genes, we performed PCR-based restriction cloning using Phusion HF Master Mix (Thermo Fisher Scientific, F-548S) and the following primers: U6-PCR-F 5′-AAAGGCGCGCCGAGGGCCTATTT-3′ (SEQ ID NO:8), U6-PCR-R 5′-TTTTTTGGTCTCCCGGTGTTTCGTCCTTTCCAC-3′ (SEQ ID NO:9), cr-RNA-R 5′-GTTCCCTGCAGGAAAAAAGCACCGA-3′ (SEQ ID NO: 10), cr-RNA-F 5′-AAAAAAGGTCTCTACCG(N)₂₀GTTTTAGAGCTAGAAATAGCAAGTT-3′ (SEQ ID NO: 11), where N₂₀ marks the sgRNA substitution site. sgRNA design was performed using a combination of tools: Synthego (https://design.synthego.com/#/), CRISPick (SpyoCas9, Hsu (2013) tracrRNA, https://portals.broadinstitute.org/gppx/crispick/public) and the Sabatini/Lander list (Addgene #1000000096). The most upstream exon-targeting guide that was represented on the most sgRNA design platforms was picked. The sgRNA sequences used are as follows: non-targeting (NT) sgRNA-5′-GCACTACCAGAGCTAACTCA-3′ (SEQ ID NO:12), sgTnfsf10-5′-ACTTCTCTCCACCCAGCTGC-3′ (SEQ ID NO:13), sgIl11ra1-5′-TTCATAGTCTACCGCTTGGC-3′ (SEQ ID NO: 14), sgIl11-5′-GCAGCCATTGTACATGCCGG-3′ (SEQ ID NO:15). U6 and crRNA PCR products were purified using QIAquick PCR Purification Kit (Qiagen, #28104) and digested using the following restriction enzymes: DpnI (NEB, #R0176S) and BsaI-HF (NEB, #R3535/R3733). The U6 fragment and the pLenti backbone were also digested with AscI (NEB, #R0558) and SbfI-HF (NEB, #R3642). The crRNA fragment was additionally digested with SbfI-HF. Digested fragments were separated by gel electrophoresis on a 0.8% agarose gel (Life Technologies, #16500100) for 20 minutes at 135V. DNA was extracted from gel fragments using QIAquick Gel Extraction Kit (Qiagen, #28704). Inserts were ligated into the lentivirus backbone overnight at 16° C. using T4 DNA Ligase Kit (NEB, #M0202L). Finally, constructs were heat shock-transformed into NEB Stable E. Coli (NEB, #C3040) at 42° C. for 30s and plated on ampicillin LB-agar selection plates (MP Biomedicals, 113002221). After overnight incubation at 37° C., single colonies were picked and plasmid DNA was isolated using QIAprep Spin Miniprep Kit (Qiagen, #27104). Successful sgRNA cloning was confirmed by Sanger sequencing (Azenta Life Science) of clones using U6-PCR-F and crRNA-rev primers described above. DNA production was scaled up using CompactPrep Maxi Kit (Qiagen, 12863). Lentiviruses were prepared in HEK293FT cells following ViraPower Lentiviral Packaging Mix protocol (Thermo Fisher Scientific, #K497500), with packaging and pseudotyping plasmids comprising pLP1 and pLP2, and pLP/VSVG, respectively. Briefly, 5×10⁶ HEK293FT cells cultured in antibiotics-free medium were transfected by use of Lipofectamine 2000 (Thermo Fisher Scientific, 11668019) complexes in Opti-MEM reduced serum medium+GlutaMAX (Thermo Fisher Scientific, 51985034). 3 μg of each helper plasmid and expression plasmid was used to form DNA-Lipofectamine complexes (12 μg DNA total, DNA: Lipofectamine ratio of 1:3), which were added dropwise to cell cultures. The following day, culture medium was replaced, and fresh medium was added. After 48 h hours, virus-containing supernatant was collected, filtered through 0.45 μm PVDF membranes and concentrated using Lenti-X Concentrator (Clontech, #631231) in accordance with the manufacturer's protocol. Final pellets were resuspended in 1/500-1/1000 of the original supernatant volume and aliquots stored at −80° C. Lentiviral titration was performed using qPCR Lentivirus Titration Kit (ABM, LV900) with undiluted, and 1:10-100 diluted samples.

Isolation, culture, and stimulation of primary mouse astrocytes. Primary murine astrocytes were isolated from neonatal brains as previously described [39]. Briefly, wild-type mouse pups aged 1-3 days were cryoanesthetized and decapitated. Whole brains were extracted, and 2-3 brains pooled per culture. After centrifugation for 5 min at 500×g, tissues were resuspended in 5 mL pre-warmed 0.25% Trypsin-EDTA (Thermo Fisher Scientific, 25200-072) and incubated at 37° C. for 10 min, followed by addition of culture medium to stop the dissociation reaction, and mechanical disaggregation by strong pipetting. The suspension was then filtered through a 70 μm nylon strainer and centrifuged 5 min at 500×g. Finally, the suspension was seeded onto T75 flasks (Falcon, 353136) that were coated at room temperature for 10 min with poly-L-lysine (Sigma-Aldrich, P4707). Mixed glial cultures were grown for at least 10 days prior to enrichment of astrocytes per the following method. Flasks were shaken at 240 rpm and 37° C. for 2-4 hours for detachment of microglia and oligodendrocytes. Next, media was washed out with PBS and 0.25% Trypsin-EDTA was used to detach astrocytes after incubation for around 5 min. Finally, astrocytes were taken up in culture medium and seeded in poly-L-lysine-coated experimental plates at 150×10³ cells per well in 24-well plates. Culture medium used for astrocytes was made of DMEM/F12+GlutaMAX (Thermo Fisher Scientific, 10565-042) with 10% fetal bovine serum (Gibco, 10-438-026), 1% penicillin/streptomycin (Thermo Fisher Scientific, 15140122), 100 mM HEPES (Gibco, 15630080) and 1× non-essential amino acids (Gibco, 11140050). Inhibition of STAT3 was performed by use of Niclosamide (BAY2353) (Selleck Chemicals, S3030) at 2 μM in dimethyl sulfoxide as vehicle (Sigma-Aldrich, 472301), since this concentration was reported to be effective but not cytotoxic [40].

In vitro co-culture of astrocytes and T cells. In vitro co-culture assays of astrocytes and T cells were performed in 24-well plates at a ratio of 1:2. Briefly, 100 or 150×10³ astrocytes per well were seeded 2 days before assay initiation and stimulated as detailed for around 16 h (overnight) prior to the addition of T cells. When blocking antibodies for TRAIL were used, they were added to astrocyte cultures for 1 h prior to T-cell addition and also in the co-culture medium. Blocking of IL-11 was performed during all of the overnight pre-stimulation as well as during the co-culture. Splenic T cells were isolated from female wild-type mice of 8-10 weeks of age and purified by magnetic selection using Naïve CD4 or CD8 T-cell Isolation Kits (Miltenyi Biotec, 130-104-453 or 130-096-543). Naïve T cells were then activated by overnight stimulation with plate-bound anti-CD3 at 10 μg/mL (bioXcell, BE0001-1-A005 mg) and 0.25 μg/mL anti-CD28 (bioXcell, BE0015-5-A005 mg) at a density of 0.3×10⁶/well. The next day, activated T cells were collected, counted, and resuspended in their respectively conditioned media before being added onto astrocytes at a 1:2 ratio. T cell assays were performed in complete RPMI medium supplemented with 0.25 μg/mL anti-CD28 to maintain activation. After 3 h of co-cultivation half of the culture medium was taken off for staining and measurement of Caspase-8 cleavage by use of Vybrant FAM Caspase-8 Assay Kit (Thermo Fisher Scientific, V35119). After a total of 8 h the rest of the culture supernatant containing T cells was taken off and stained to detect Caspase-3/7 activation using CellEvent Caspase-3/7 Green Flow Cytometry Assay Kit (Thermo Fisher Scientific, C10740).

Functional blocking with monoclonal antibodies. For functional blocking assays the following monoclonal antibodies were used: TRAIL blocking antibody (Thermo Fisher Scientific, 16-5951-85) and its respective rat anti-IgG2a kappa Isotype control (Thermo Fisher Scientific, 16-4321-82) were used at 5 μg/ml. Human/mouse cross-reactive anti-IL11 blocking antibody (R&D Systems, MAB218) and its respective mouse anti-IgG2a kappa Isotype control (Thermo Fisher Scientific, 14472485) were used at 10 μg/ml.

Flow cytometry. For flow cytometry, single cell suspensions were obtained as described above. A small aliquot (usually 1/10th of sample) was taken out prior to staining and diluted 1:10-20 in PBS. This aliquot was run on the flow cytometer using the plate reader setting, which allows analysis of defined sample volumes and therefore back-calculation of absolute cell numbers per sample. The rest of the sample was first stained with LIVE/DEAD Fixable Aqua Dead Cell Stain Kit for 405 nm excitation (Thermo Fisher Scientific, L34966) at 1:2500 dilution and anti-CD16/CD32 Fc block (BD Biosciences, 553142), then washed in 0.5% BSA and 2 mM EDTA pH 8.0 in PBS (FACS buffer), and then stained extracellularly with a mix of fluorescently-labeled antibodies in FACS buffer for 30 min in the dark at 4° C., followed by another wash with FACS buffer. For intranuclear staining, cell pellets were fixed for 20 min at room temperature in fixation buffer with a FoxP3 transcription factor staining kit (eBioscience, 00-5523-00), followed by a washing step, and staining for 30 min at room temperature in the provided wash buffer. Finally, samples were washed with FACS buffer and analyzed on a BD FACSymphony Analyzer (BD Biosciences). Data was analyzed using FlowJo (v10.8.1). Antibodies used for T-cell analysis were: BUV496 anti-SLAMF6 (BD Biosciences, 750046), BUV563 anti-CD11b (BD Biosciences, 741242), BUV661 anti-CD45 (BD Biosciences, 612975), BUV737 anti-PD-1 (BD Biosciences, 749306), BUV805 anti-LAG-3 (BD Biosciences, 748540), BUV650 anti-CD3 (BD Biosciences, 740530), BV711 anti-TIGIT (BD Biosciences, 744214), BV750 anti-TNF (BD Biosciences, 566365), BV786 anti-TIM3 (BD Biosciences, 747621), FITC anti-CD8a (Biolegend, 100804), PerCP-Vio 700 anti-DR5 (Miltenyi Biotec, 130-102-478), PE anti-CTLA4 (Biolegend, 106306) or BV421 anti-CTLA4 (Biolegend, 106312), PE/Dazzle 594 anti-IL-10 (Biolegend, 505034), PE/Cy5 anti-IL2 (Biolegend, 503824), PE/Cy7 anti-CD4 (Biolegend, 100422), AF700 anti-CD39 (Invitrogen, 56-0391-82), APC/Cy7 anti-IFNγ (BD Biosciences, 561479). GL261-specific CD8⁺ T cells were stained with an APC-conjugated H-2D (b) tetramer loaded with GARC-1₇₇₋₈₅ peptide (AALLNKLYA) (NIH Tetramer Core) was used. For detection of PE-conjugated HSV-1-specific CD8⁺ T cells H-2Kb tetramer with HSV-1 glycoprotein B peptide SSIEFARL (Immudex, JD2670) was used. Antibodies used for detection of apoptosis: BUV737 anti-CD11b (BD Biosciences, 612800), BV421 anti-CD11a (BD Biosciences, 740020), BV605 anti-CX3CR1 (Biolegend, 149027), BV650 anti-CD3 (BD Biosciences, 740530), BV711 anti-CD8a (BD Biosciences, 752634), BV785 anti-Ly6C (Biolegend, 128041), Cellevent Caspase-3/7 Flow Cytometry Assay Kit (Thermo Fisher Scientific, C10740) as described below, PerCP-Vio 700 anti-DR5 (Miltenyi Biotec, 130-102-478), PE/Cy7 anti-CD4 (Biolegend, 100422), APC anti-CD45 (Thermo Fisher Scientific, 17-0451-83), APC/Cy7 anti-CD49d (Biolegend, 103635). Antibodies used for measuring TRAIL expression and phospho-STAT3 in TdTomato+astrocytes were: BUV661 anti-CD45 (BD Biosciences, 612975), BUV737 anti-CD11b (BD Biosciences, 612800), BV421 anti-CD11a (BD Biosciences, 740020), BV605 anti-CX3CR1 (Biolegend, 149027), BV785 anti-Ly6C (Biolegend, 128041), PE/Cy7 anti-TRAIL (Biolegend, 109311), APC anti-pSTAT3 (Y705) (Thermo Fisher Scientific, MA5-36902), APC/Cy7 anti-CD49d (Biolegend, 103635).

Fluorescence-activated cell sorting. Single cell suspensions were stained extracellularly with a mix of antibodies in FACS buffer for 30 min at 4° C. as described above. All antibodies were diluted 1:100 in FACS buffer. Prior to sorting, cell suspensions were filtered through tubes with 35 μm cell strainer caps (Falcon, 352235). Antibodies used for sorting were: BV421 anti-CD11a (BD Biosciences, 740020), BV605 anti-CX3CR1 (Biolegend, 149027), BV650 anti-CD11b (BD Biosciences, 563402), BV711 anti-CD8a (BD Biosciences, 752634), BV785 anti-Ly6C (Biolegend, 128041), FITC anti-CD3 (Biolegend, 100204), PE/Cy7 anti-CD4 (Biolegend, 100422), APC anti-CD45 (Thermo Fisher Scientific, 17-0451-83), APC/Cy7 anti-CD49d (Biolegend, 103635). CD4 T cells were sorted as Live⁺ CD45⁺ CD11b⁻ CD3⁺ CD4⁺. CD8 T cells were sorted as Live⁺ CD45⁺ CD11b-CD3⁺ CD8⁺ cells. Microglia were sorted as Live⁺ CD45⁺ CD11b⁺ Ly6C^{low}/−CX3CR1⁺ CD49d^{low}, CD11a^{low}. Monocytes were sorted as CD45⁺ CD11b⁺ Ly6C^{high} CD49d^{high} CD11a^{high}. Cells were sorted using a FACSAria IIU (BD Biosciences). Astrocyte sorting from Aldh111^{Cre-ERT2/TdTomato} astrocyte reporter mice was performed by gating Live⁺ CD45⁻ CD11b⁻TdTomato⁺.

Flow cytometric analysis of T cells. For flow cytometry of T cells, magnetic separation was used to negatively select CNS-infiltrating T cells by use of the Pan T-cell Isolation Kit II mouse (Miltenyi Biotec, 130-095-130). T cells were then reactivated in a complete RPMI containing 50 ng/ml Phorbol 12-myristate 13-acetate (PMA) (Sigma Aldrich, P8139), 1 μM Ionomycin (Sigma Aldrich, I3909-1ML), and 1:1500 of each GolgiStop and GolgiPlug (BD Biosciences, 554724), and 1:100 BUV395 anti-LAMP-1 (BD Biosciences, 565533) for 4 h at 37° C. T cell culture medium was composed of RPMI 1640 (Gibco, 11875119) supplemented 10% FBS, 1% penicillin/streptomycin, 50 μM 2-metcaptoethanol (Sigma-Aldrich, M6250), 1% non-essential amino acids (Thermo Fisher Scientific, 11140050), 10 mM HEPES (Thermo Fisher Scientific, 15630130), 1 mM Sodium Pyruvate (Thermo Fisher Scientific, 11360070), and 1× GlutaMAX (Thermo Fisher Scientific, 35050061). T cells were then incubated with 50 nM Dasatinib (Selleck Chemicals, S1021) to improve tetramer staining and 1:200 FcBlock (BD Biosciences, 553142) for 20 min at 37° C. prior to addition of fluorescently labeled MHC tetramers at a final dilution of 1:50 in 2% FBS for staining at room temperature for 1 h in the dark. Washing steps were performed with FACS buffer and centrifugation at 600×g for 5 min at 4° C. Next, surface markers were stained in FACS buffer for 30 min at 4° C., followed by fixation and permeabilization with BD Cytofix/Cytoperm (BD Biosciences, 554715) for 20 min at 4° C. according to the supplied protocol. Intracellular markers were then stained with an antibody solution prepared in permeabilization buffer. Finally, stained T cells were stored at 4° C. in FACS buffer until analysis.

Apoptosis analysis. Apoptosis was analyzed by flow cytometry by use of the Cellevent Caspase-3/7 Flow Cytometry Assay Kit (Thermo Fisher Scientific, C10740), staining samples with Caspase-3/7 detection agent for 30 min at 37° C. To ensure consistent staining, SYTOX AADvanced Dead Cell Stain was added to each tube exactly 1 min prior to measurement by flow cytometry. To measure death-receptor-induced apoptosis, the Vybrant FAM Caspase-8 Assay Kit (Thermo Fisher Scientific, V35119) was used per manufacturer's protocol.

RNA Extraction and Real-Time qPCR.

For quantitative analysis of gene transcription from cultured cells cellular RNA was extracted by use of RNeasy Kit (Qiagen, 74004). Briefly, cells were lysed in 350 μL RLT buffer and stored at −80° C. until extraction following manufacturer's instructions. RNA was transcribed to cDNA using the High-Capacity cDNA Reverse Transcription Kit (Life Technologies, 4368813). Quantitative measurement of gene expression was performed by qPCR using Taqman Fast Universal PCR Master Mix (Life Technologies, 4367846), MicroAmp Optical 384 well plates (Thermo Fisher Scientific, 43-098-49) and a ViiA 7 Real-Time PCR System (Applied Biosciences). Taqman probes used in this study were: ActB (Mm02619580_g1), Tnfsf10 (Mm01283606_ml). qPCR data were analyzed by the ddCt method by normalizing the expression of each gene for each replicate by the average of the housekeeping gene (ActB) and then normalizing treatment group to control group to determine a relative change.

Bioinformatic RNA-seq analysis. For bulk RNA-seq the raw fastq files are processed using Kallisto [41], which aligned the read pairs to GRCm38 reference genome and quantified the gene expression of each gene based on Gencode vM37 [42]. Then tximport [43] package is used to organize the output from Kallisto and the differential expression analysis was performed using DESeq2 (v4.3). For scRNA-seq analysis, sequenced reads from pooled samples were trimmed and demultiplexed using Trim Galore (v0.6.5), assigned to their associated cell barcodes and unique molecular identifiers (UMIs), and aligned to the ENSEMBL reference genomes for mouse and human. Drop-seq software was used to correct bead synthesis errors and quantify gene expression [36]. The Seurat package (v4.3) was used to normalize count matrices, remove doublets, correct batch effects, and integrate samples into one data set. The SCTransform function was used to normalize and scale expression matrices, stabilize variance, regress out variance due to mitochondrial genes, and identify the 2000 most variable genes. Next, Principal Component Analysis (PCA) was performed to identify the parameters for dimensionality reduction. For clustering, we used the Louvain algorithm with multilevel refinement. We applied the MAST algorithm for differential expression analysis between clusters as part of the FindAllMarkers function of Seurat. Differential expression analyses between specific groups were performed with FindMarkers. For pathway analysis, gene set enrichment analysis (GSEA) (Broad Institute, v4.3.2) was run using a pre-ranked list with the average expression log 2 (FC) as the ranking metric. Curated gene signatures from Molecular Signatures Database (MSigDB) v2023.1 were used for identification of relevant pathways. We show analysis of Hallmark gene sets (h.all) and a large collection of curated gene sets (c2.all), which can be found on the MSigDB website. Furthermore, Ingenuity Pathway Analysis (IPA) (Qiagen) was performed using gene IDs and average expression log 2 (FC), filtering to include 2,000 to 3000 genes at most. We performed core analysis and made use of pathway enrichment and upstream regulator tools. Any signature scores shown were calculated using the AddModuleScore function in Seurat. Differential abundance testing for mouse scRNA-seq data was conducted using miloR (v1.8.1), constructing a k-nearest neighbors (KNN) graph and assigning cells to a neighborhood (k=20, d=9). Next, differential abundance testing was performed in a generalized linear model framework between tumor and control samples using testNhoods (robust=TRUE, fdr.weighting=“graph-overlap”). Grouping of neighborhoods was performed using the groupNhoods function (overlap=1, max.lfc.delta=5). Groups were then assigned to scRNA-seq clusters and spatialFDR as well as log FC were plotted. For TCGA bulk RNA-seq cell type deconvolution, the gene expression data of TCGA-GBM was downloaded from the GDC Data Portal (National Cancer Institute) and the de-convolution algorithm BayesPrism was used to extract cell type-specific expression profiles from each sample [44]. For imputation of TRAIL expression, the CollecTri database of the transcriptional regulatory networks and the decoupleR package were used. For visualization Seurat, dittoSeq (v4.3) or ggplot2 (v3.4.3) was used. Volcano plots were made with EnhancedVolcano (v3.17). Additional thanks to github.com/PeeperLab for sharing for sharing the replotGSEA function. All R-based analyses were performed on RStudio (v4.3.0) with R (v4.3.1).

ELISA. Protein-level detection of mouse IL-11 in TCM was performed via enzyme-linked immunosorbent assay (ELISA) using the Mouse IL11 ELISA Kit (Thermo Fisher Scientific, EMIL11) following the manufacturer's protocol and read out on GloMax Explorer Multimode Microplate Reader (Promega) at 450 nm.

GBM survival analysis. Survival analysis of TCGA data was performed using cBioPortal dataset Glioblastoma Multiforme (TCGA, Firehose Legacy) and patient survival data was downloaded for 166 samples with transcriptional data available (mRNA expression z-scores relative to all samples (log RNA Seq V2 RSEM). Statistical analysis and survival curves were produced in R using the survminer package. In addition, the Gepia2 database was used for survival analysis based on gene or signature expression in the ‘GBM’ dataset, and to detect correlation with similarly expressed genes [46].

Human primary astrocyte culture. Primary human fetal astrocytes were isolated as previously described [47]. Second trimester fetal brain tissues were obtained from Centre Hospitalier Universitaire Sainte-Justine, Montreal, Canada or from the Birth Defects Research Laboratory, University of Washington, Seattle, USA with maternal written consent and under local ethic boards' approval. Isolation of glial cells was carried out through mechanical and chemical digestion. Primary fetal astrocytes were maintained in Dulbecco's Modified Eagle Medium (DMEM)/F12 (Thermo Fisher Scientific, 11320033) with 10% fetal bovine serum (FBS) (Wisent Bio Products, 080-450), 1% penicillin/streptomycin (Thermo Fisher Scientific, 15140122) and 1% GlutaMAX (Thermo Fisher Scientific, 35050061) and cultured at 37° C. with 5% CO2. For in vitro stimulation conditions, human primary astrocytes were seeded at 80,000 cells per well. The cell media was aspirated, cells were washed with 37° C. PBS and the fresh, appropriately conditioned media was then added into wells. Human astrocytes were stimulated for 6 hours with U87 TCM (see above), 50 ng/ml recombinant human IL-11 (PeproTech, 200-11). At the end of the treatment, total RNA was isolated and transcribed and analyzed by qPCR as described above.

Immunofluorescence staining of tissue sections. Following perfusion with ice cold PBS, mouse brains were dissected and fixed in 4% Paraformaldehyde solution (Electron Microscopy Sciences, 15714S) at 4° C. overnight, then dehydrated in 30% Sucrose (Sigma-Aldrich, S0389) for a week. Fixed brains were then grossly dissected to embed in Tissue-Tek O.C.T. Compound (Sakura Finetek USA, 4583) and stored at −80° C. Tissue blocks were cut on a Leica Cryostat CM1950 (Leica) at 8 μm and mounted on Tissue Path Superfrost Plus Gold Slides (Thermo Fisher Scientific, 15-188-48) and frozen at −20° C. Immunofluorescence staining was performed by permeabilization with 1×BD Perm/Wash Buffer (BD Biosciences, 554723) for 10 mins at room temperature, followed by blocking of unspecific binding with serum-free protein block (Agilent, X090930-2) for 10 minutes at room temperature. Primary and secondary antibodies were diluted in BD Perm/Wash buffer and tissue slides incubated with primary staining solution at 4° C. overnight, then washed with BD Perm/Wash buffer 3 times, and then incubated with secondary staining solution for 1 hour. After staining, ProLong Gold Antifade Mountant (Thermo Fisher Scientific, P36930) was added onto the tissue, and the section was sealed with a High Precision 1.5H cover slips (Azer Scientific, ES0107052) and nail polish (Electron Microscopy Sciences, 72180). For phospho-STAT3 staining, tissue permeabilization was performed with 0.5% Triton-X (Sigma-Aldrich, T8787-100ML) and 4% BSA in PBS for 1 h at room temperature. Primary antibody staining was performed in the same solution overnight at room temperature, followed by washing in 1×BD Perm/Wash Buffer, secondary staining for 1 h at room temperature in 1×BD Perm/Wash Buffer, followed by DAPI (Sigma-Aldrich, D9542-1 MG) staining for 5 mins. Mounting and slide sealing were performed similarly for all stainings. Confocal fluorescence microscopy was performed on a Zeiss LSM710 (Zeiss) at 20× magnification. Antibodies used were: mouse anti-GFAP-AF488 (eBioscience, 53-9892-82), Chicken anti-GFAP (Abcam, ab4674), donkey anti-Chicken-AF405 (Jackson ImmunoResearch, 703-475-155), rabbit anti-phospho-STAT3 (Cell Signaling Technology, 9145T), donkey anti-Rabbit-AF647 (Thermo Fisher Scientific, A31573), goat anti-TRAIL (Invitrogen, PA5-47073), donkey anti-Goat-AF568 (Thermo Fisher Scientific, A11057), rat anti-CD3-FITC (eBioscience, 11-0032-80), rabbit anti-Cleaved Caspase-3 (Asp175) (Cell Signaling Technology, 9661T).

Multiplex immunofluorescence human GBM. Multiplexed immunofluorescence and immunohistochemistry labelling was performed in analogy to the iterative indirect immunofluorescence imaging (4i) protocol by Gut et al23. In short, formalin-fixed paraffin-embedded tissue sections of 2 μm thickness were mounted on adhesive glass slides in an adaptable chamber system (Ibidi sticky slides, Gräfelfing, Germany) before deparaffinization and heat-induced antigen retrieval at pH 9. As described previously 23, each immunofluorescence imaging cycle consisted of blocking, indirect immunofluorescence staining and DAPI staining (Carl Roth, Karlsruhe, Germany), imaging on a Keyence BZ-X810 compact fluorescence microscope (Keyence Corporation, Osaka, Japan) in imaging buffer, and antibody elution. After five iterations, subsequent immunohistochemistry for EGFRvIII and GFAP as well as hematoxylin counterstaining were appended (adhering to standard operating procedures of the Institute of Neuropathology in Freiburg, Germany) on the same sections, and stepwise imaging performed in brightfield modus at identical location on the same microscope. Images of immunofluorescence labelling and immunohistochemistry labelling (with inverted greyscale intensity) were aligned and integrated, based on nuclear staining in DAPI and hematoxylin. This approach resulted in a 15-plexed source file for downstream analysis of each sample, created in Image J (Version: 1.53t with Java 1.8.0_345 (64-bit)). Antibodies use in cycle 1 used were: GFAP (Dako, Z0334), CD3 (BioRad, MCA1477T), DR4 (R&D Systems, MAB347); cycle 2: IL-11 (LSBio, LS-C177617-50), OLIG2 (Biotechne, AF2418), DR5 (R&D Systems, MAB6311); cycle 3: IL11RA (Abcam, ab125015), TRAIL (R&D Systems, AF375), IBA1 (Synaptic Systems, 234 308); cycle 4: pSTAT3 (Cell Signaling Technology, 9145); cycle 5: Cleaved Caspase-3 (Cell Signaling Technology, 9664); cycle 6: EGFRVIII (Biozol, GTX02628).

Immunofluorescence image analysis. Image analysis was conducted using the QuPath application (v0.3.2 with Java 16.0.2). Analysis of multiplexed immunofluorescence images of human GBM are described first. In brief, comprehensive training of automated analysis tools was carried out across samples. First, regions of interest were restricted to parenchymal/tumoral regions (including endothelial structures but excluding erythrocyte-filled luminal areas and other areas of clearly unspecific antibody binding by use of a pixel classifier). Next, automated segmentation was performed using the ‘Watershed cell detection’ algorithm referring to DAPI nuclear signal. Automated cell type classification was performed using the ‘train object classifier’ algorithm, whereby positive or negative cells were manually selected until the accuracy of the classifier was sufficient. GFAP was used to identify tumor cells and astrocytes, IBA1 for myeloid cells, and CD3 for T cells. To identify TRAIL⁺ astrocytes, we additionally trained an object classifier to detect cells positive for GFAP and TRAIL and excluded tumor cells by use of thresholding in the EGFRvIII signal and the nuclear-to-cytoplasm ratio based on a healthy control sample. Apoptotic T cells were classified by training of an object classifier for cells positive for CD3 and cleaved Caspase-3. The distance to the nearest TRAIL+astrocyte was calculated by use of the ‘detect centroid distances 2D’ function in QuPath. The number of specific detections was then divided by the surface area of the analysed image (post exclusion of unspecific signals via pixel classification). Detection measurements and calculated distances were exported, converted to fcs format (flowCore and analyzed in FlowJo (v10.9.0) and R. For analysis of immunofluorescence images of mouse brain, we similarly used the ‘cell detection’ and ‘train object classifier’ algorithms and quantified number of detections over analyzed area. Data points are biological replicates, except for distance measurements, where each detected cell was plotted from n=4 GBM patient specimens.

Promoter activity assay. Human TRAIL promoter activity was assayed by use of Secrete-Pair Gaussia Luciferase Assay (Genecopoeia, LF062). Briefly, Hep G2 cells were transiently transfected with a human TNFSF10 promoter reporter plasmid (Genecopoeia, TRAIL-HPRM37867-PG02) by use of Lipofectamine 3000 (Thermo Fisher Scientific, L3000008) at a DNA to Lipofectamine ratio of 1 μg:1.5 μL. 24 hours after transfection, cells were stimulated with U87 TCM or recombinant human IL-11 (PeproTech, 200-11) for 24 hours. Supernatants were collected and any cell debris centrifuged off at 500×g for 5 min. Luciferase activity assays were performed following the manufacturer's protocol and analyzed on GloMax Explorer Multimode Microplate Reader (Promega). High sensitivity buffer was used to detect the effect of IL-11 on TRAIL expression, while U87 TCM was measured by use of the stable detection buffer.

Generation of HSV-1ª-TRAIL Anti-TNF-related Apoptosis-inducing Ligand (TRAIL) Mouse Monoclonal Antibody [clone: 75411.11] (VWR, Radnor, PA, USA) was sequenced by LC-MS/MS at Rapid Novor (Kitchener, Ontario, Canada). VH and VL regions were assembled in a scFv format fused to a mIgG1 Fc domain to generate the TRAIL-blocking single-chain antibody. Sequence of the VH region:

(SEQ ID NO: 1)
QVTLKESGPGILQPSQTLSLTCTFSGFSLTTHGMGVGWIRQPAGKGLEW
LANIWWDDDKFHSPSLKNRLTISKDTSNNQAFLRITNVDTADAAIYYCA
RISWERAYYFDYWGQGVMVTVSSAETTAPSVYPLAPGTALKSNSMVTLG
CLVKGYFPEPVTVTWNSGALSSGVHTFPAVLQSGLYTLTSSVTVPSSTW
SSQAVTCNVAHPASSTKVDKKIVPRECNPCGCTGSEVSSVFIFPPKTKD
VLTITLTPKVTCVVVDISQNDPEVRFSWFIDDVEVHTAQTHAPEKQSNS
TLRSVSELPIVHRDWLNGKTFKCKVNSGAFPAPIEKSISKPEGTPRGPQ
VYTMAPPKEEMTQSQVSITCMVKGFYPPDIYTEWKMNGQPQENYKNTPP
TMDTDGSYFLYSKLNVKKETWQQGNTFTCSVLHEGLHNHHTEKSLSHSP
G;

sequence of the VL region:

(SEQ ID NO: 2)
DVQMTQSPSFLSASVGERVTLSCRASQNVDTYLDWYQQKLGEAPKLLIY
NTNNLHTGIPSRFSGSGSGTDYTLTISSLQPEDVATYFCLQRNRWLTFG
SGTKLEIKRADAAPTVSIFPPSMEQLTSGGATVVCFVNNFYPRDISVKW
KIDGSEQRDGVLDSVTDQDSKDSTYSMSSTLSLTKVEYERHNLYTCEVV
HKTSSSPVVKSFNRNEC.

The expression construct encoding the single-chain antibody was cloned between the CAG promoter and the rabbit β-globin pA and inserted using gateway recombination in the UL3/UL4 intergenic region of ONCR-142 DNA in a bacterial artificial chromosome (BAC). The excision of the BAC vector sequence was achieved by Cre-loxP recombination. BAC-removed viruses were plaque-purified in the Vero-SF cell line prior to expansion, purification by centrifugation, determination of titer, and vialing.

Oncolytic virus therapy. All virus stocks were thawed and prepared immediately before dosing and kept on ice until administration. For survival and efficacy studies, treatment and control groups were randomized based on tumor bioluminescence signal at day 6 after GL261-mNectin1 tumor implantation such that both groups displayed similar average signal. On day 7, mice were injected with 3×10⁶ plaque forming units (PFU) HSV-1 intratumorally. Animals were monitored daily for clinical signs and body weight, and given dietary support as needed. Mice were euthanized in accordance to experimentation protocols once a body weight decrease of 20% occurred. Survival differences were calculated by log-rank (Mantel-Cox) test.

Spatial transcriptomics reanalysis. Publicly available 10× Visium spatial transcriptomics data from human GBM was analyzed using the SPATA2 R package⁹¹. Spatially-weighted Pearson correlation analysis of our pathways of interest is shown for sample UKF265_T. For Ivy Glioblastoma Atlas Project data analysis, expression data for genes of interest was downloaded and heatmaps replotted using R.

Statistical analysis. GraphPad Prism 10 (GraphPad Software Inc., v10.0.2) was used for most bar graph visualizations and to calculate significant differences. Sample sizes (n) denote biological replicates, and not technical repeated measurements of the same sample. For in vitro studies, replicates indicate independent biological samples randomly allocated into experimental groups. For in vivo studies, replicates indicate individual mice on a C57Bl/6J background allocated randomly to experimental groups. No statistical methods were used to pre-determine sample sizes. Sample size was chosen based on previous experiments. Difference testing between groups was done using a two-sided student's t-test or a one-way ANOVA followed by Tukey's post-hoc test as indicated in the figure legends for each experiment. For mouse survival studies, Kaplan-Meier curves are shown, and the Log-rank (Mantel-Cox) test was performed to assess significance of survival differences between groups. A P value <0.05 was considered significant.

### Example 1. Immune Suppression by Brain Tumor-Associated Astrocytes

### Single-Cell Characterization of Human and Mouse GBM-Associated Astrocyte Subsets (FIG. 1)

To analyze mechanisms involved in the immune regulation by astrocytes in the GBM microenvironment, we first performed single-cell RNA sequencing (scRNA-seq) of human and mouse glioma samples (FIG. 1a). We obtained clinical specimens of resected GBM tumors or control tissue from autopsies and performed RNA-seq at the single-nucleus level. For the identification of non-malignant cell types, we relied on analysis of copy-number variations in chromosomes 7 and 10 and on canonical markers and cell type signatures. We detected differences in the proportion of different astrocyte subsets in our human data, as well as in an external scRNA-seq dataset of GBM-associated astrocytes (FIG. 1b-c, f-g). To investigate the heterogeneity of GBM-associated astrocytes deeply, we made use of the syngeneic, immunocompetent model of glioma GL261-Luc2 by implanting 1×10⁵ cells into the striatum of astrocyte inducible reporter mice Aldh11/TdTomato 5 fluorescently labeling all astrocytes upon Tamoxifen administration (FIG. 1a). Tumor burden was assessed via bioluminescence imaging at days 7 and 14. On day 15, tumor-bearing mice were sacrificed, and brains were dissected to separate left (distal) and right (tumor-bearing) hemispheres for microfluidic scRNA-seq of FACS-sorted TdTomato+astrocytes (FIG. 1d-e). Analysis of the higher depth scRNA-seq data from mouse astrocytes revealed 13 distinct clusters, suggesting extensive heterogeneity of cell states in the homeostatic and diseased state (FIG. 1d), which was less visible in human data due to technical limitations single-nuclei sequencing as well as limited availability of resected tumor material. The transcriptomic profile of several astrocyte clusters was influenced by the presence of GBM, as indicated by enrichment of these cell states specifically in tumor-bearing hemispheres, or, inversely, the relative decrease in these cell states in the context of malignancy (FIG. 1e). Notably, cluster 0 was increased by 40% in tumor-bearing mice.

### A TRAIL-Expressing Astrocyte Subset in the Tumor Microenvironment (FIG. 1-2)

We previously reported the presence of an IFNγ-induced astrocyte subpopulation that plays an anti-inflammatory role in the context of MS by inducing apoptosis of autoreactive T cells via Tumor necrosis factor-related apoptosis-inducing ligand (TRAIL) [11]. We detected an increase in Tnfsf10 transcripts in mouse cluster 0 cells (FIG. 1h). In human data, we observed higher expression of TNFSF10 in astrocytes from cancer samples (FIG. 1h), as well as a correlation of TNFSF10 expression with earlier recurrence (FIG. 1j).

Having observed increased astrocytic expression of Tnfsf10 in scRNA-seq data in mouse and humans, we hypothesized that this astrocyte state could also be relevant in the context of GBM, having the opposite effect on disease outcome by worsening the anti-tumor immune response. Using our Aldh11/TdTomato astrocyte reporter mouse strain, we investigated these astrocytes further using flow cytometry and detected increased surface expression of TRAIL on astrocytes in tumor-bearing mice as compared to sham-injected mice (FIG. 2a, b). To gain mechanistic insight about the transcriptional program driving TRAIL+ astrocytes in GBM, we performed bulk RNA-seq of sorted TRAIL+ astrocytes, revealing significant transcriptional changes in this astrocyte subset (506 genes upregulated, 117 genes downregulated) (FIG. 2c). Pathway analysis of this transcriptional signature revealed that STAT3 signaling appears to be highly involved in the regulation of this TRAIL+ astrocyte subset in GBM, as opposed to STAT1 regulation as previously observed (FIG. 2d,e). Overall, these results suggested that a TRAIL-expressing astrocyte subset exists and is upregulated in the context of GBM, and that this activation state could be regulated by STAT3.

### Astrocyte-Mediated Killing of T Cells Via TRAIL is a Mechanism of Immunosuppression (FIG. 3)

To functionally investigate this astrocyte subset in tumor-bearing mice, we performed in vivo astrocyte-specific genetic deletion of Tnfsf10 using lentivirus-delivered CRISPR-Cas9 (TRAIL-KD) followed by implantation of GL261-Luc2 cells (FIG. 3a). Compared to astrocytes infected with a non-targeting guide RNA, mice undergoing astrocyte-specific TRAIL-KD exhibited longer survival in the GL261-Luc2 model of glioblastoma (FIG. 3b), prompting us to further investigate the effect of this knockdown on T cells in the TME. We examined apoptotic responses in T cells at day 15 following GL261 implantation and found a decreased proportion of apoptotic CD4+ and CD8+ T cells in TRAIL-KD mice (FIG. 3c). In addition, the fraction of tumor-specific CD8+ T cells was increased, as measured by tetramer staining loaded with the GL261-endogenous neoantigen GARC-1 (FIG. 3d). The absolute number of tumor-targeted CD8+ T cells showed a trend to be increased (FIG. 3d). Overall, these data suggest enhanced T cell survival and function following astrocyte-specific TRAIL-KO, which significantly affect long-term tumor clearance.

To replicate the astrocyte-T cell interaction observed in the GBM TME in vivo, we established an in vitro co-culture assay of primary mouse astrocytes and T cells (FIG. 3e). Astrocytes were stimulated with GL261 tumor-conditioned medium (TCM) prior to addition of either CD4+ or CD8+ activated T cells. TCM pre-treated astrocytes increased the activation of caspase-3/7 and caspase-8 in CD4⁺ and CD8⁺ T cells in a TRAIL-dependent manner, indicative of DR-driven apoptosis (FIGS. 3f-i). These findings suggest that GBM-secreted factors drive TRAIL⁺ astrocytes, which limit tumor-specific immunity by inducing extrinsic apoptosis in T cells.

### GBM-Derived IL-11 Induces TRAIL Expression Via STAT3 Signaling (FIG. 4)

Having observed enriched STAT3 signaling in TRAIL+ astrocytes following bulk RNA-seq (FIG. 2d), we hypothesized that STAT3 was a central regulator of TRAIL induction in astrocytes. Indeed, astrocyte stimulation with TCM strongly induced expression of Tnfsf10 transcripts, which was fully abrogated following STAT3 inhibition with BAY2353 (FIG. 4a-b). Notably, in the context of TCM stimulation, STAT1 appeared to be less critical as a regulator of Tnfsf10 expression, as its inhibition only partially reduced the effect (FIG. 4c). Multi-omics studies of GBM combining transcriptional characterization with quantification of protein phosphorylation similarly suggested a correlation between phospho-STAT (Y705) abundance and TNFSF10 expression (FIG. 4d). Overall, our data suggests a link between STAT3 activity and TRAIL expression.

We performed a computational screen to identify secreted factors constitutively expressed by GL261 cells that could induce STAT3 signaling. IL-11 appeared to be a likely candidate, as the expression of its specific receptor chain Il11ra1 was congruent with the mouse astrocyte subset which most expressed Tnfsf10 (FIG. 4e). The relevance of IL-11 in the GBM TME has been previously reported [25, 26]. Indeed, we confirmed substantial levels of secreted IL-11 in TCM from GL261 cells by ELISA (FIG. 4f). Furthermore, when stimulating astrocytes with TCM in the presence of anti-IL-11 neutralizing antibodies, the induction of Tnfsf10 was reduced (FIG. 4g). Recombinant murine IL-11 alone at levels comparable to those found in GL261 TCM was sufficient to induce Tnfsf10 expression in a STAT3-dependent manner (FIG. 4h-i). Finally, higher IL-11 expression correlated with significantly shorter survival in patients from a The Cancer Genome Atlas (TCGA) cohort (FIG. 4j). When using our astrocyte-targeted lentivirus-delivered CRISPR-Cas9 construct to delete Il11ra1 in vivo, we observed somewhat extended survival (FIG. 4k). Altogether, these data establish a direct link between tumor-derived IL-11 and the STAT3-regulated induction of TRAIL expression in GBM-associated astrocytes, suggesting that tumor-derived IL-11 modulates astrocytes in the TME to cement immunosuppression and promote tumor progression.

### Example 2. Therapeutic Blockade of TRAIL Using Engineered Oncolytic Viruses Extends Survival

Oncolytic viruses have long been investigated as a therapeutic strategy for cancer [27]. Oncolytic viruses are attractive tools for cancer immunotherapy because of their ability to selectively lyse tumor cells [28-32]. In addition, oncolytic viruses can be engineered to express immunomodulatory molecules, with the goal of reversing the immunosuppressive characteristics of the TME [33]. More recently, genetic engineering of oncolytic viruses has been proposed as an adaptable treatment modality to specifically interfere with or stimulate specific pro-tumor mechanisms in the TME. We engineered an oncolytic herpes simplex virus-1 (oHSV) HSV-1 ONCR-142, KOS strain, with the capability of infecting GL261 cells by equipping GL261 cells with the endogenous HSV-1 entry receptor mNectin-1, using an oncolytic herpes simplex virus-1 (HSV-1) vector optimized for safety and potency [34] (Tables 2 and 3). To maximize infection and replication rates we used a gB-D285N, A549T double mutant to increase the efficiency of viral envelope fusion post-receptor engagement. The KOS strain contains a US9-R58Stop mutation that prevents anterograde transport in neurons, which supports the safety profile in the case of neuronal infection. To further ensure safety and tolerability of this treatment, a microRNA attenuation cassette was incorporated so as to protect non-malignant cells in the brain from viral infection and lysis. Finally, in order to disrupt the T cell-deletive function of TRAIL+ astrocytes in a therapeutic manner, we integrated a transgenic construct encoding TRAIL-blocking monoclonal antibodies (oHSV-TRAIL) (FIG. 5a) comprising a single chain antibody derived from mAb clone 75411, ensuring its capacity to block TRAIL-driven induction of apoptosis (FIG. 5b, 6a).

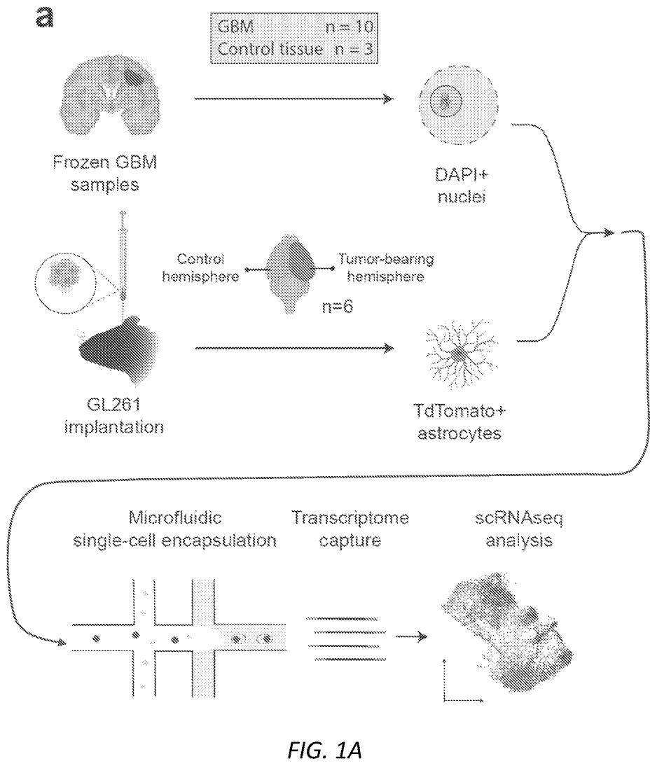

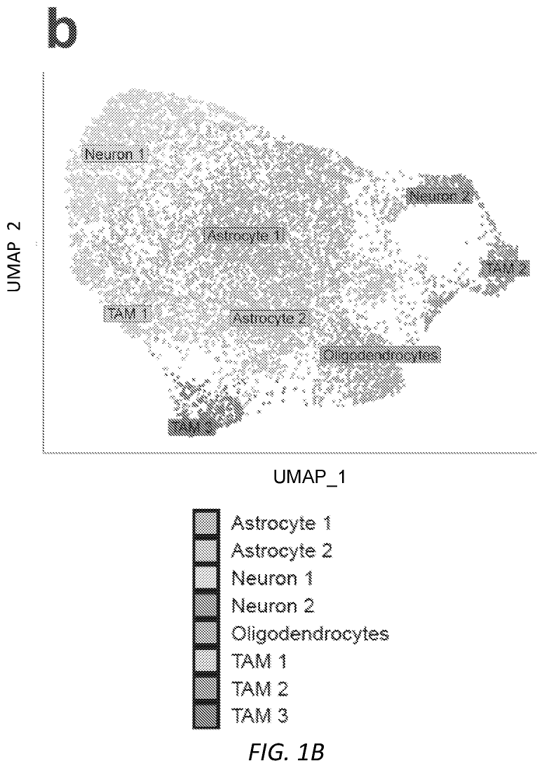

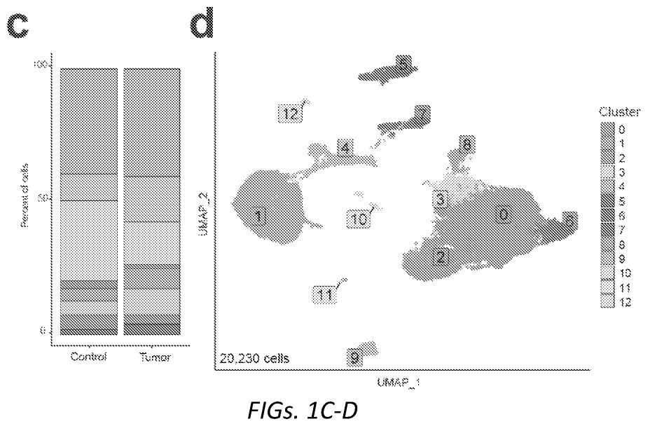

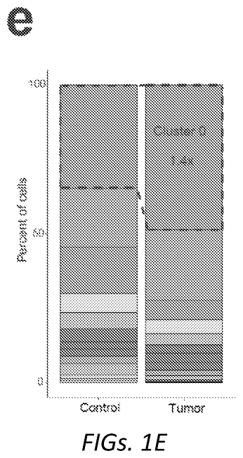

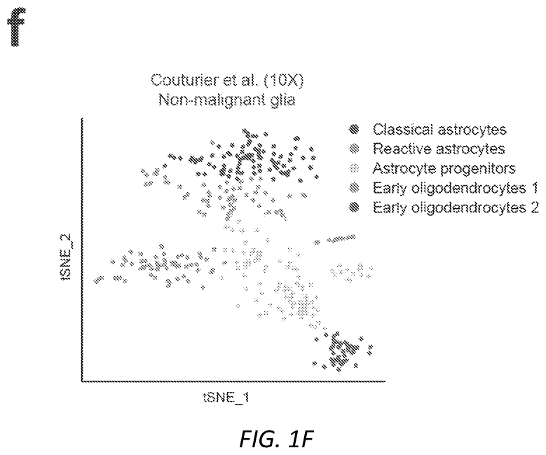

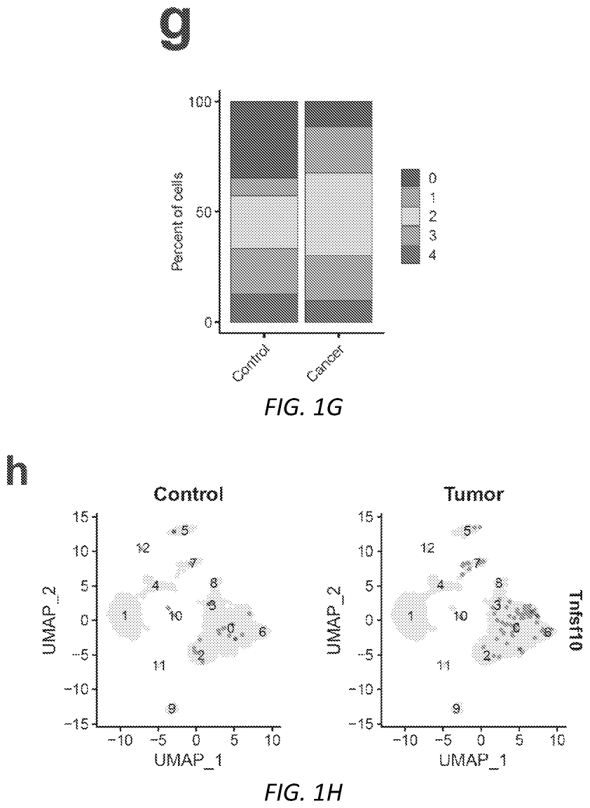

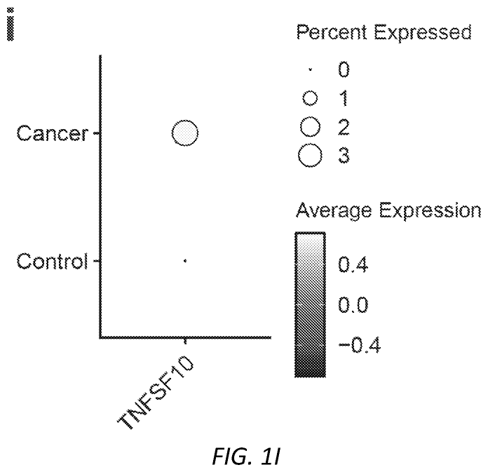

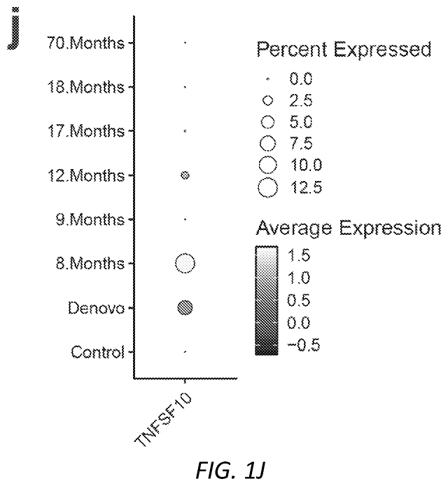

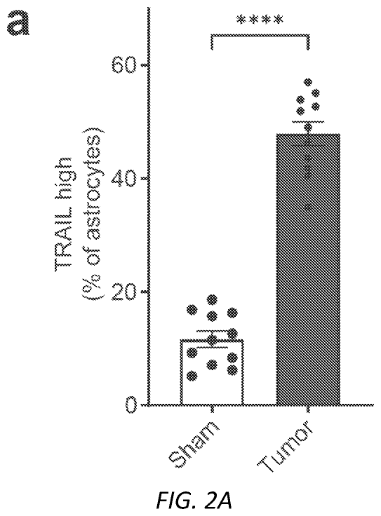

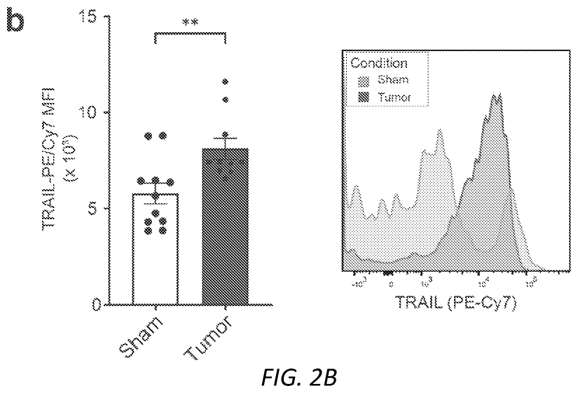

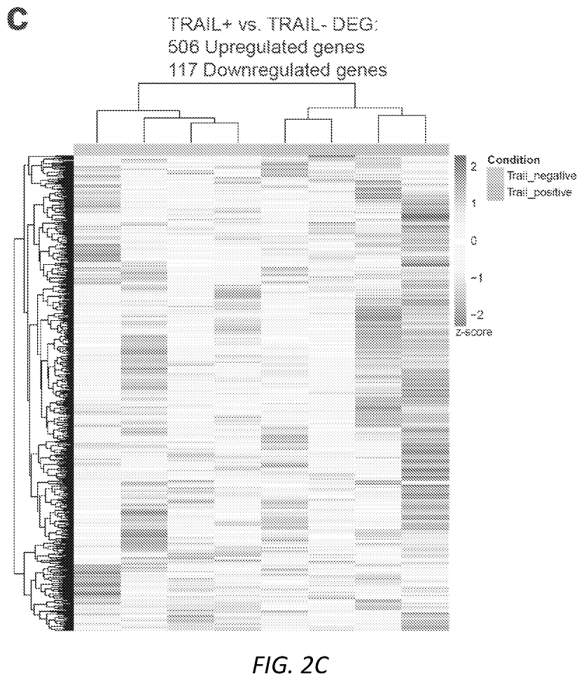

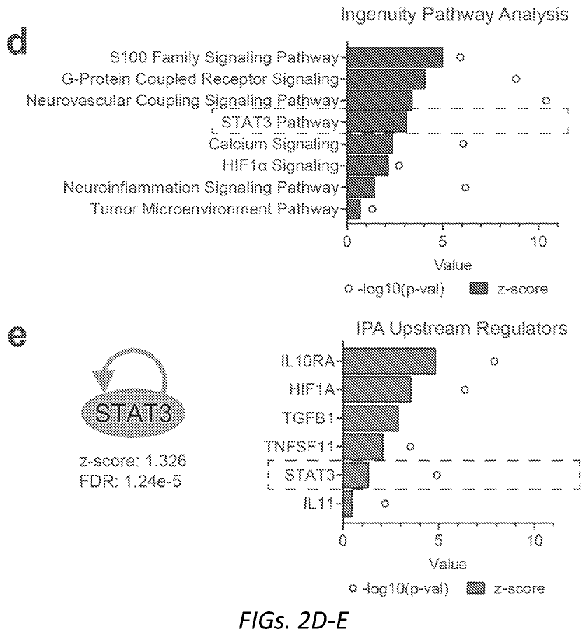

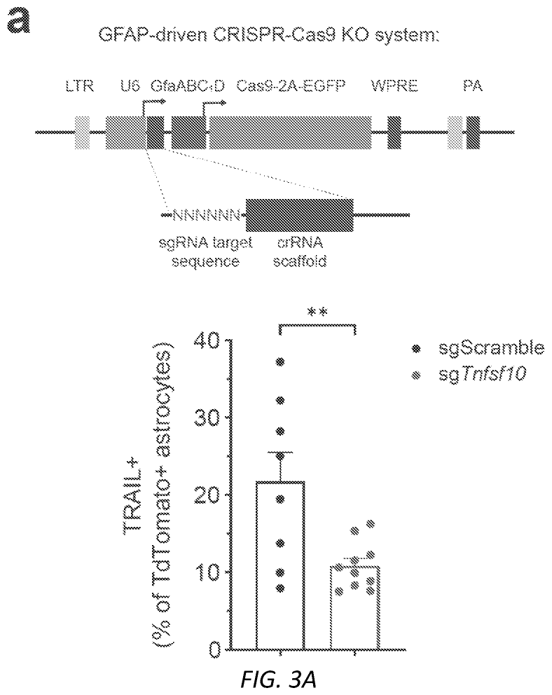

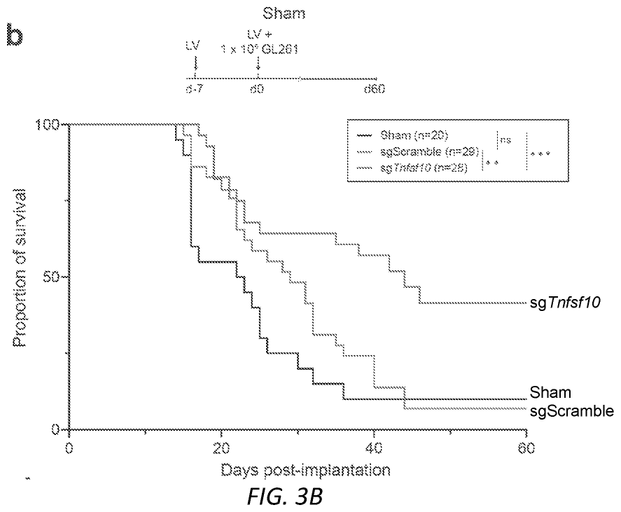

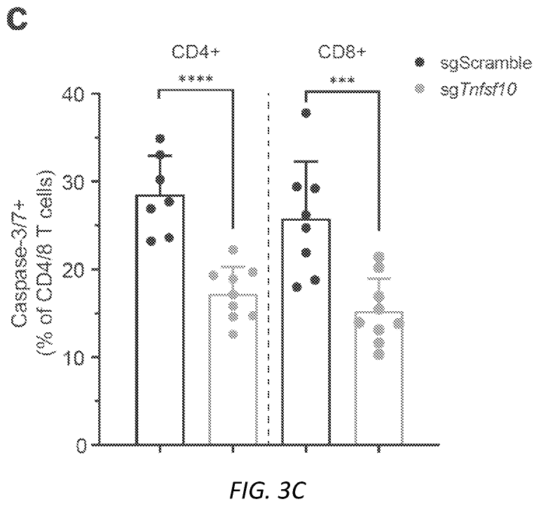

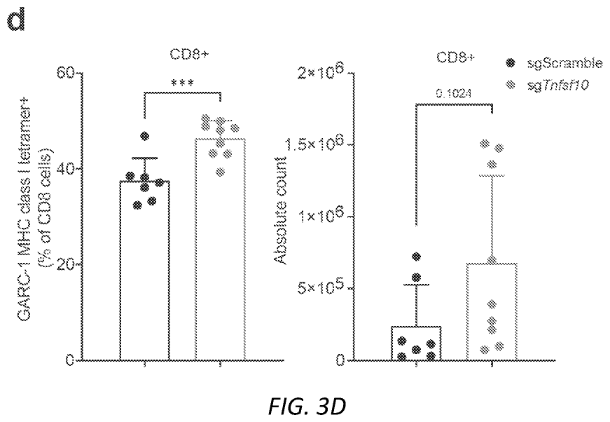

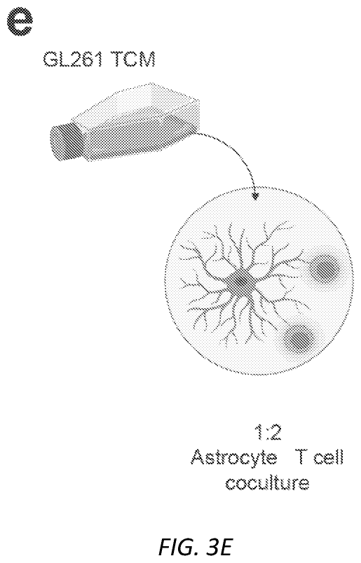

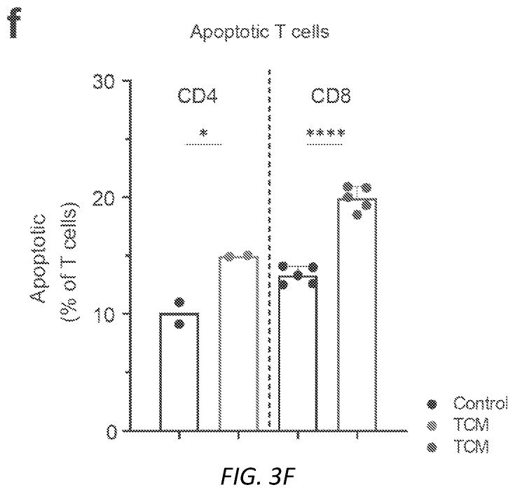

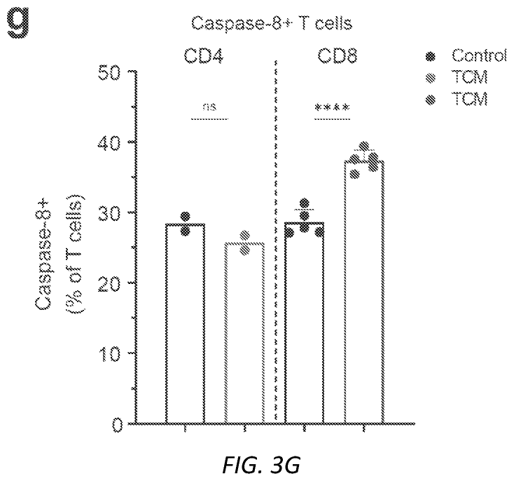

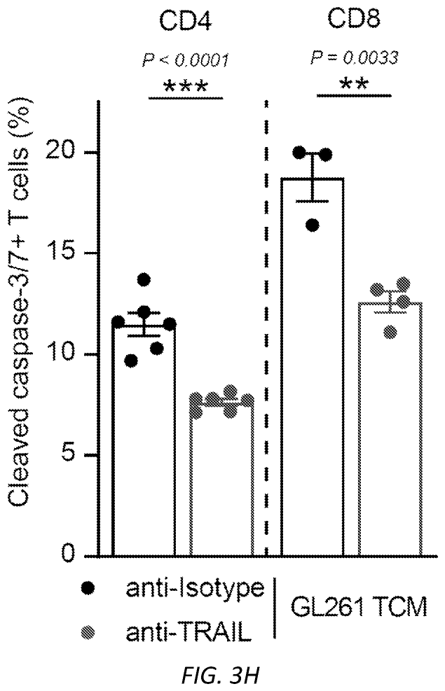

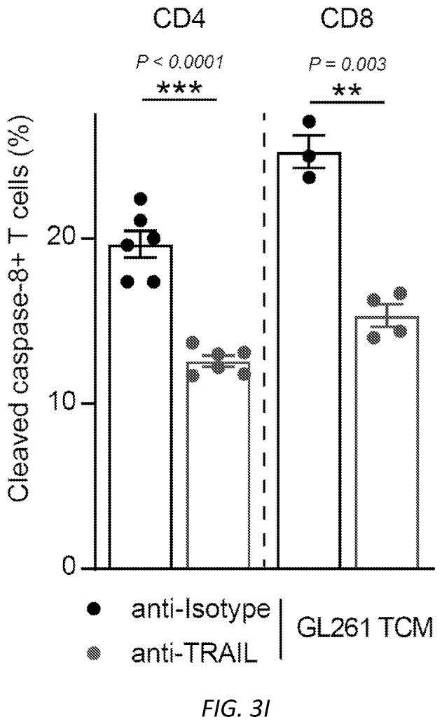

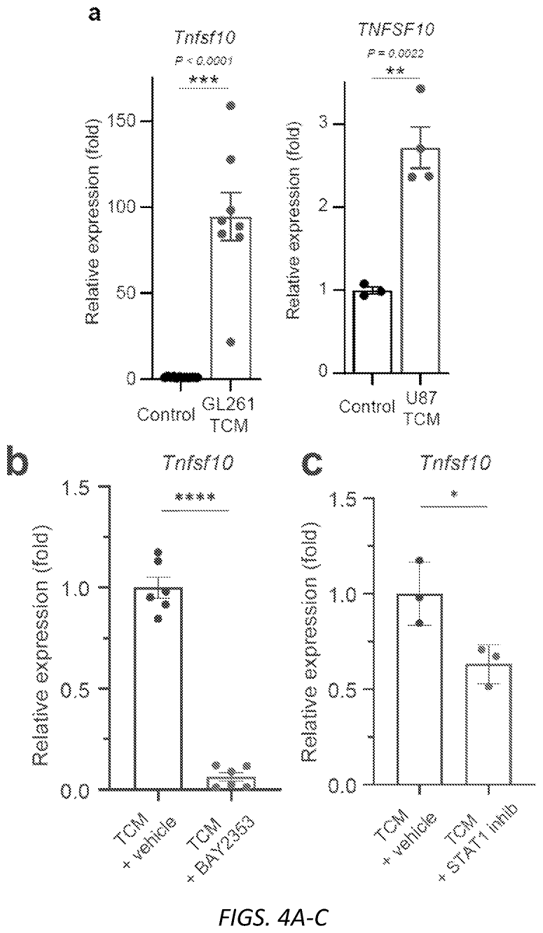

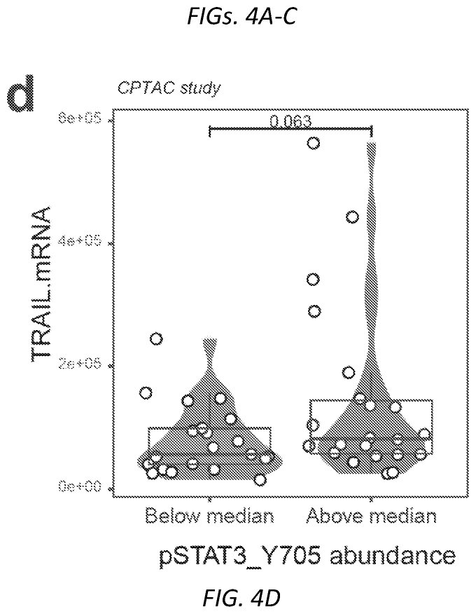

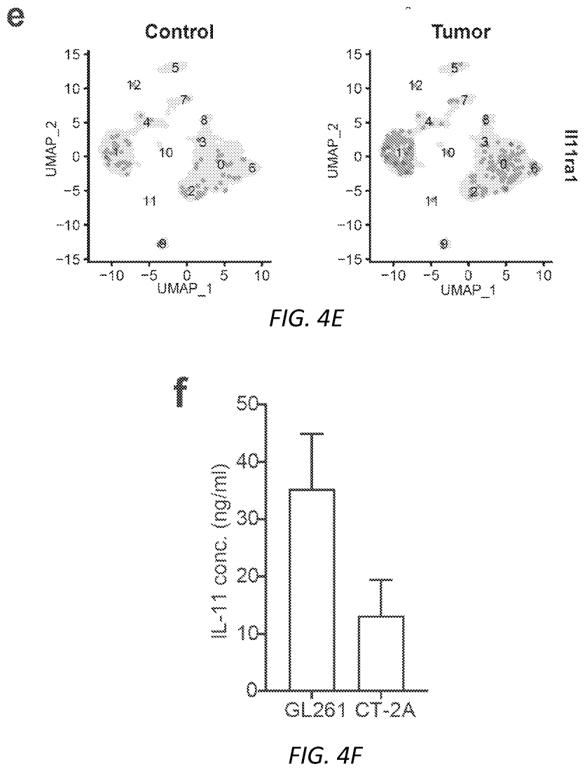

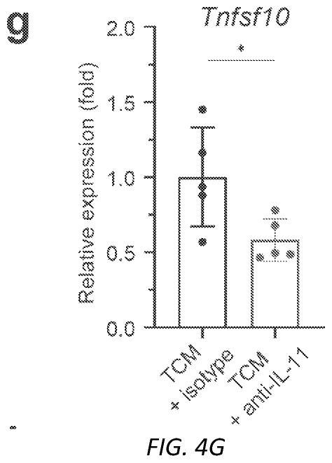

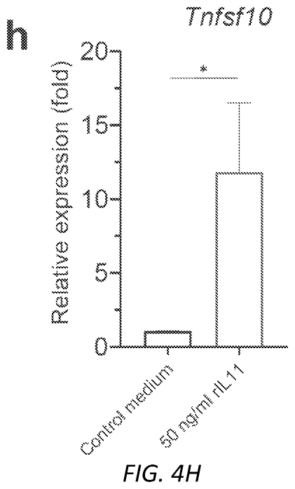

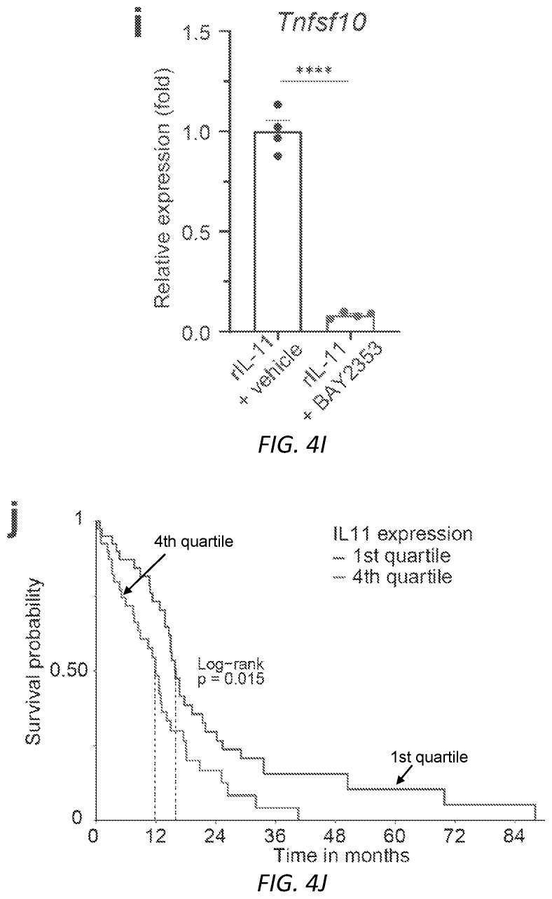

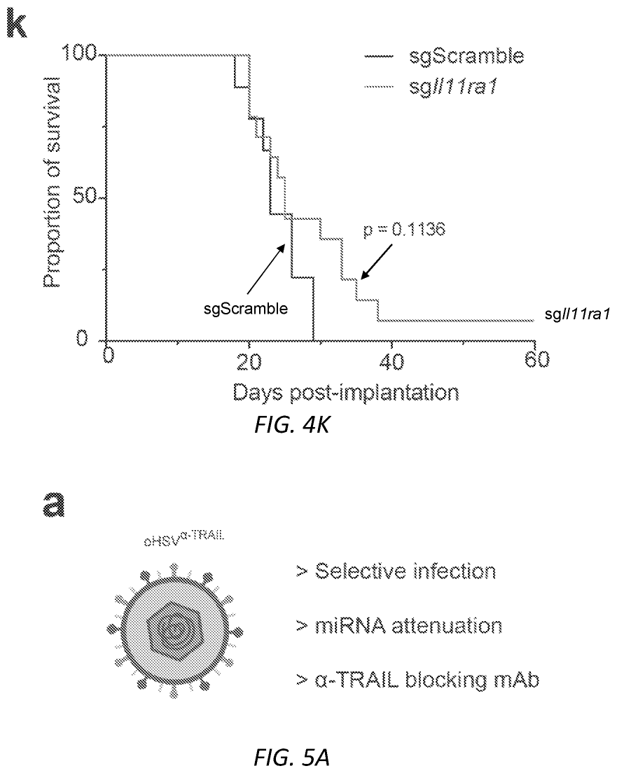

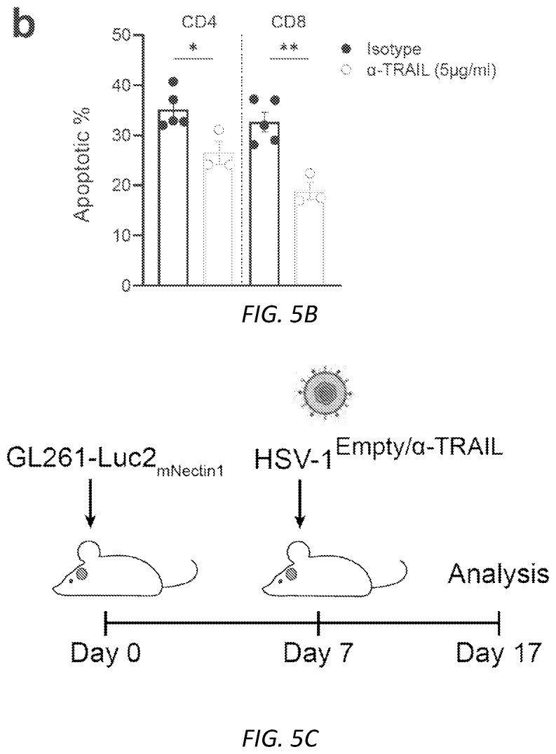

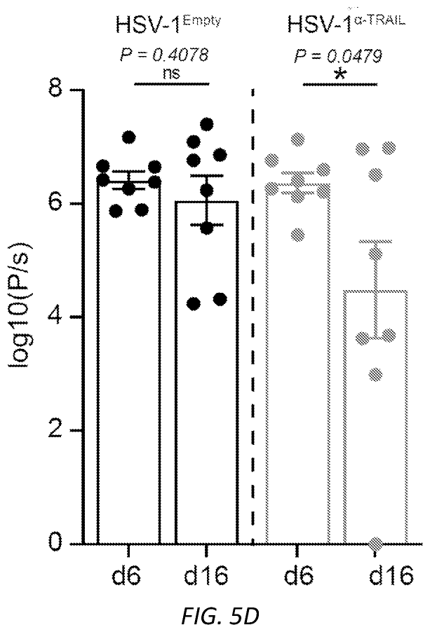

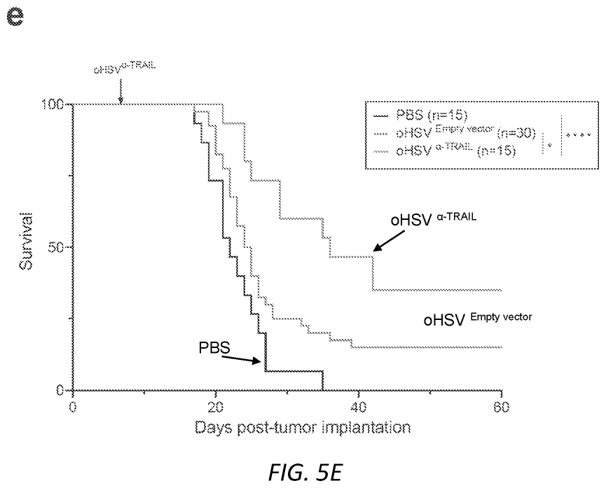

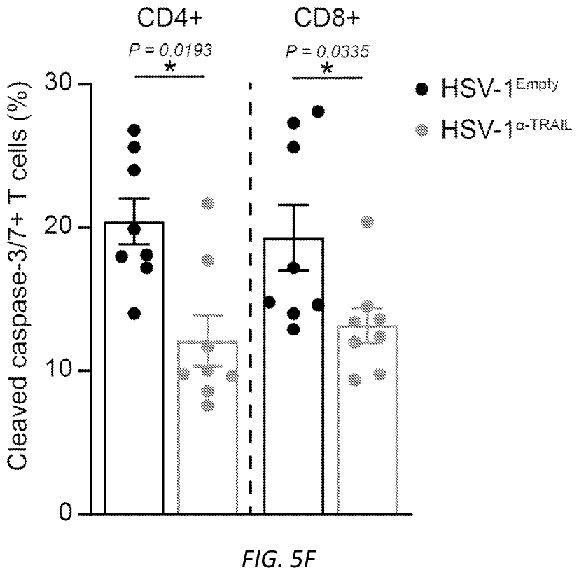

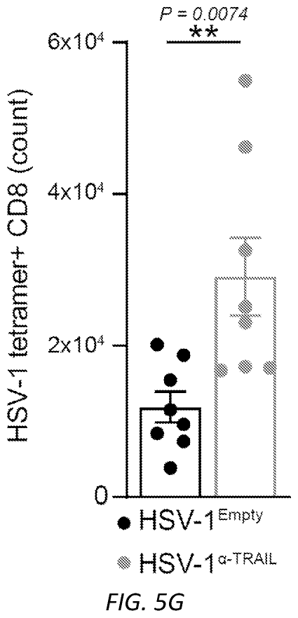

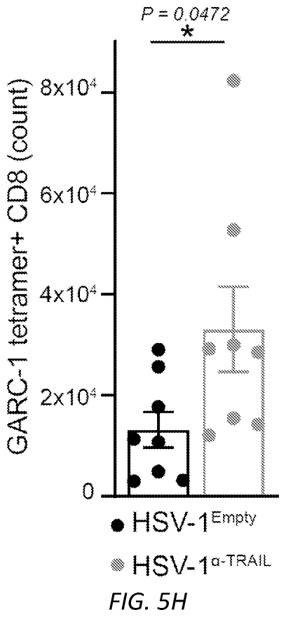

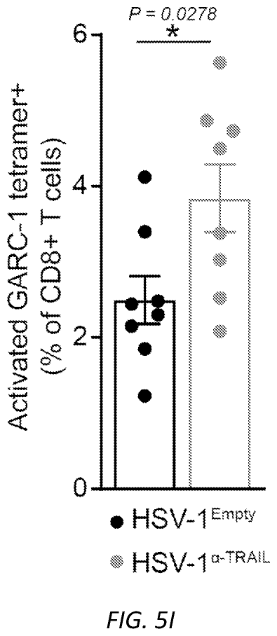

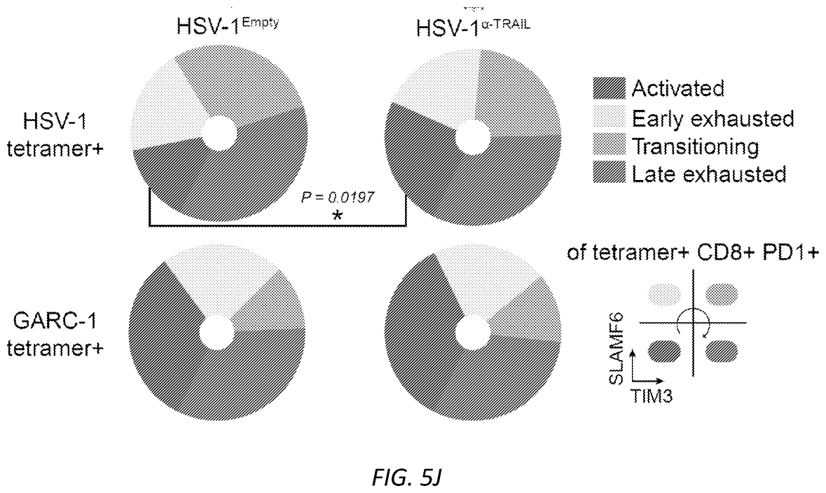

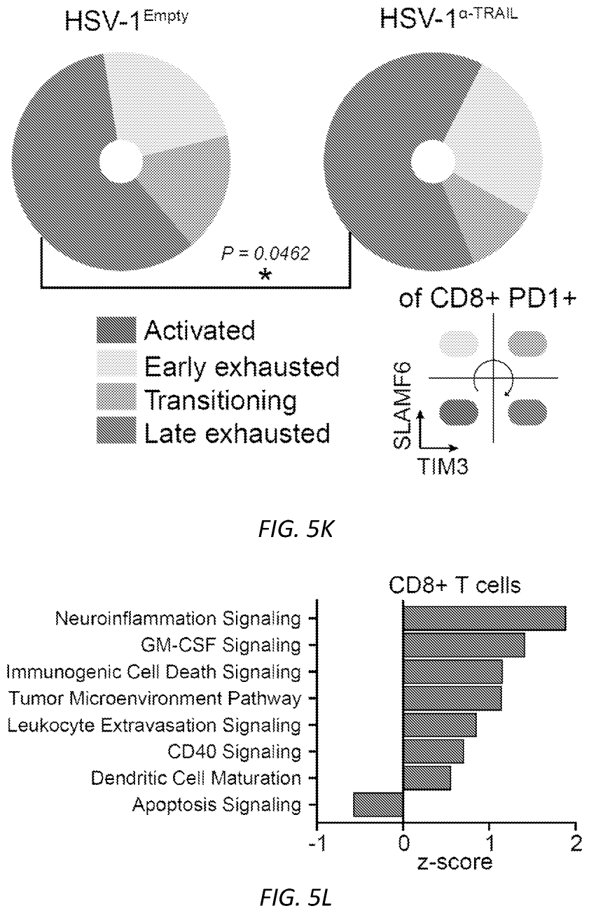

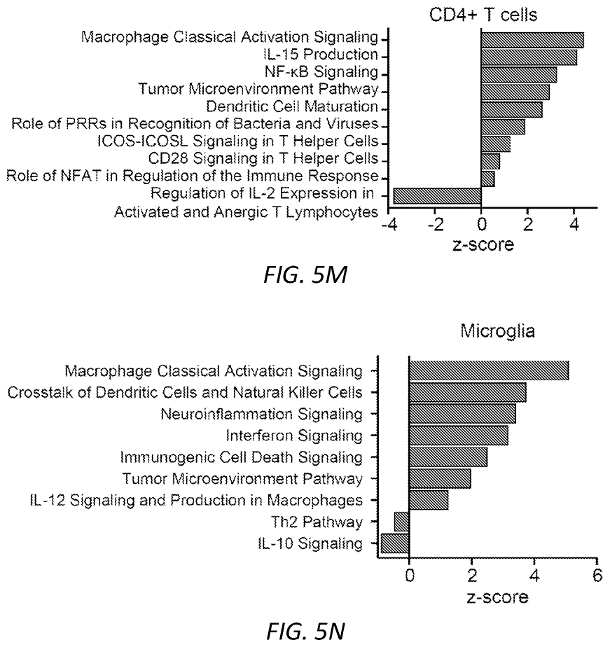

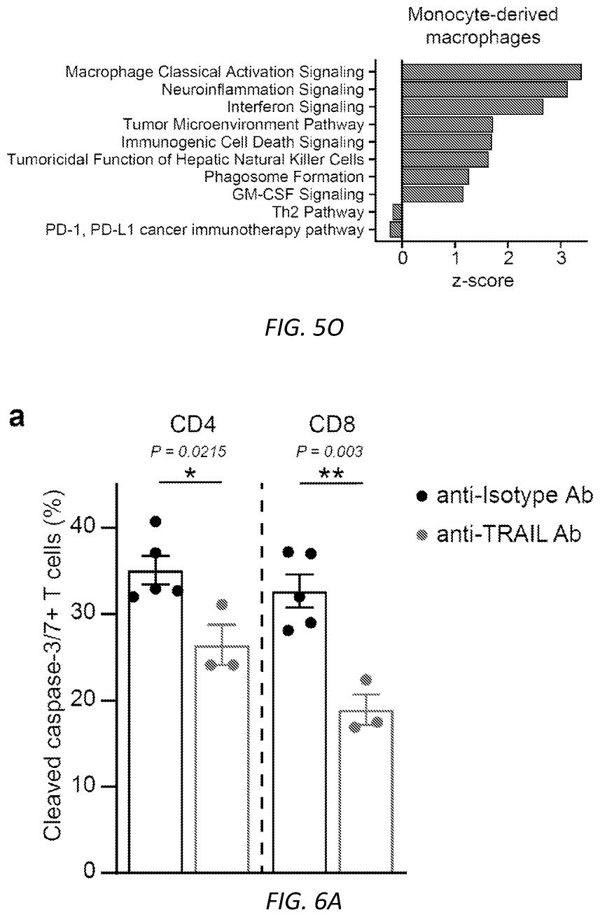

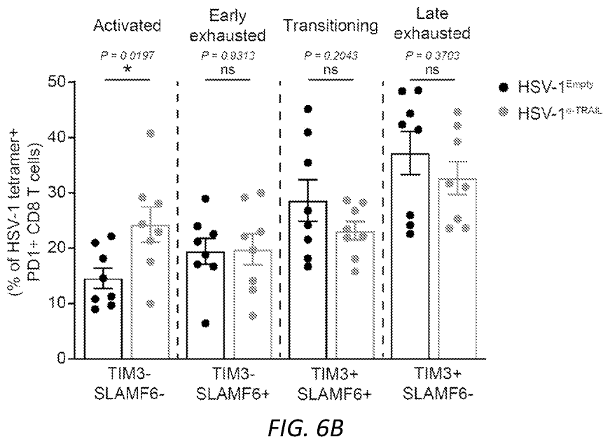

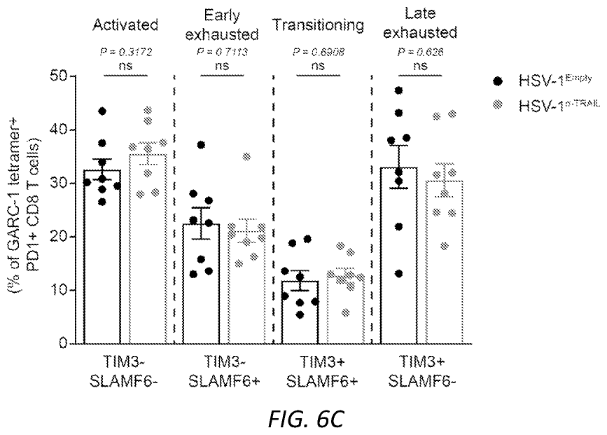

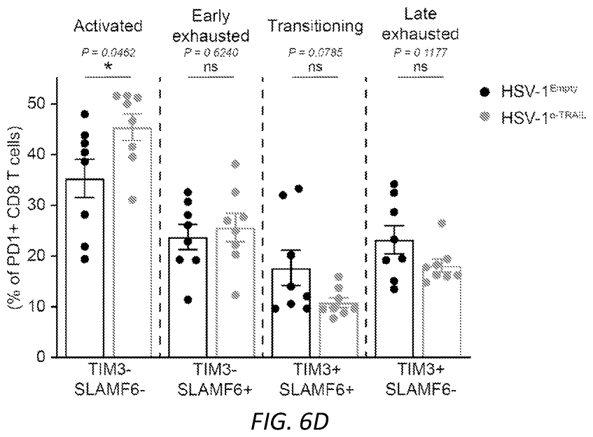

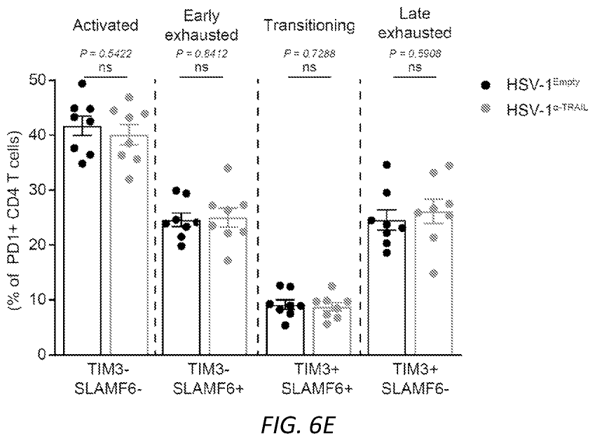

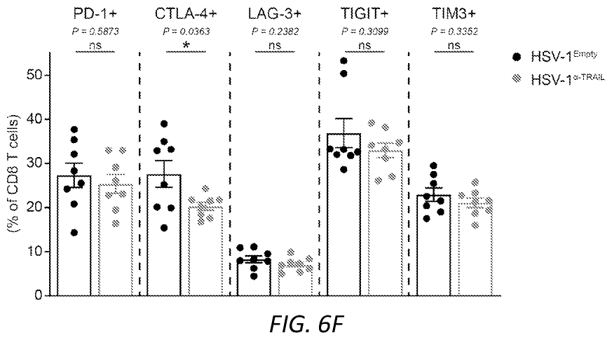

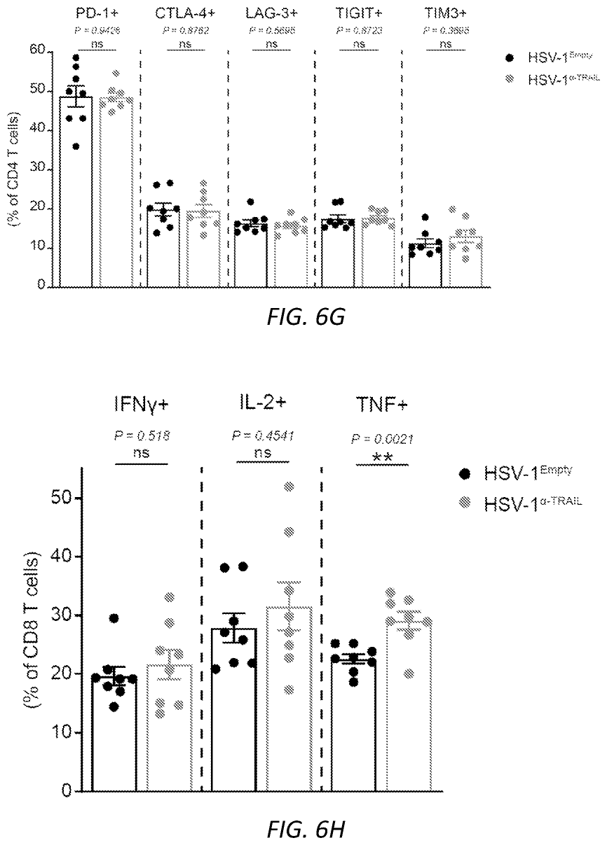

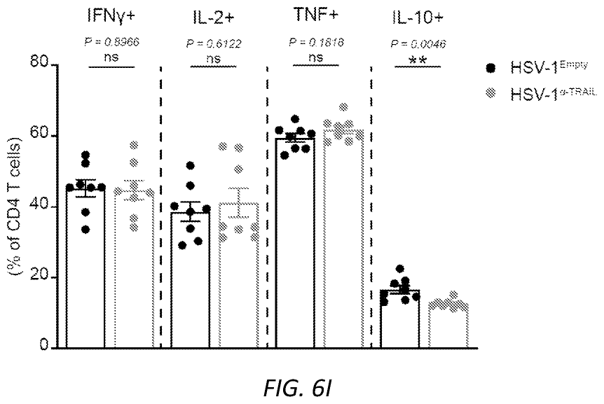

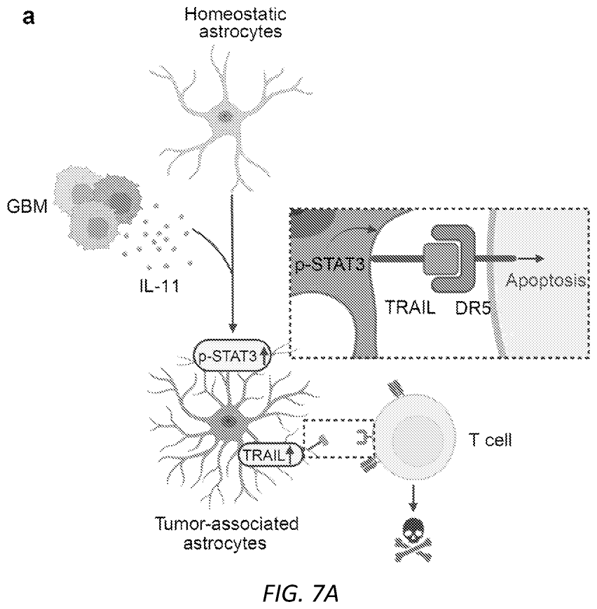

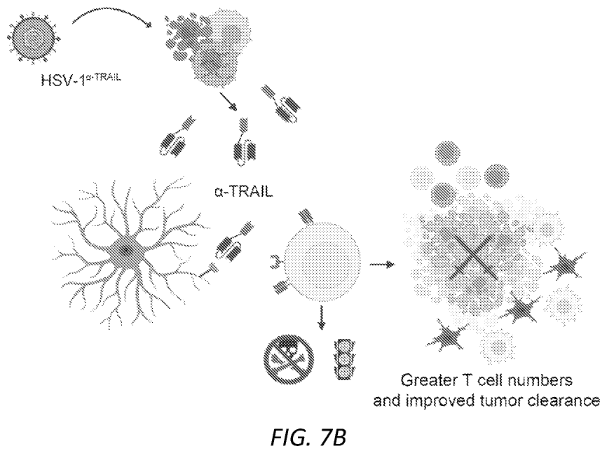

To test the anti-tumor efficacy of this oncolytic virus therapy, tumor-bearing immunocompetent B6 mice received intratumoral administration of the engineered oHSV^{α-TRAIL} virus 7 days after orthotopic implantation of GL261 cells expressing the HSV entry receptor mNectin-1 and randomization of tumor load among treatment groups (FIG. 5c). Compared to a virus lacking exogenous expression of α-TRAIL antibodies, HSV-1^{α-TRAIL} administration resulted in the reduction of tumor load quantified on day 16 by in vivo bioluminescence imagining (FIG. 5d) and significantly extended survival over 60 days of monitoring (median survival 36 d in oHSV^{α-TRAIL}-treated vs. 24.5d in empty virus-treated mice) (FIG. 5e). We went on to characterize the TME of oHSVa-TRAIL-treated mice at day 17 by flow cytometry, detecting a decrease in the fraction of apoptotic CD4 and CD8 T cells (FIG. 5f), suggesting alleviated immunosuppression from TRAIL+ astrocytes. Moreover, HSV-1^{α-TRAIL} administration increased the number of tumor- and virus-specific CD8⁺ T cells in the TME detected using peptide-loaded MHC-I tetramers (FIGS. 5g,h). Importantly, HSV-1^{α-TRAIL} treatment increased the abundance of tumor-reactive activated PD-1⁺ SLAMF6⁻TIM3⁻CD8⁺ T cells in mice treated with HSV-1^{α-TRAIL} (FIGS. 5i-k, FIGS. 6b-e), while decreasing the expression of the exhaustion marker CTLA-4 (FIGS. 6f,g). Indeed, TNF production was boosted in TME-infiltrating CD8⁺ T cells isolated from HSV-1^{α-TRAIL}-treated mice, while IL-10 production in CD4⁺ T cells was diminished (FIGS. 6h,i).

Finally, we characterized by RNA-seq the effects of HSV-10-TRAIL treatment on T cells, and microglia- or monocyte-derived TAMs in the GL261 TME. We detected reduced apoptotic signaling and increased pro-inflammatory functions in CD8⁺ T cells of HSV-1^{α-TRAIL}-treated mice (FIG. 51). Moreover, HSV-1^{α-TRAIL} administration upregulated the expression of transcriptional modules in CD4⁺ T cells associated with Th1 responses and classical macrophage activation (FIG. 5m). In agreement with these findings, HSV-1^{α-TRAIL} treatment activated phagocytic, tumoricidal and immunogenic signaling pathways in microglia- and monocyte-derived TAMs, while reducing IL-10 signaling (FIGS. 5n-o). HSV-1^{α-TRAIL} treatment also boosted bidirectional TAM-T-cell cross-talk, as suggested by the increased expression of genes involved in T-cell recruitment (Cxcl9, Cxcl10, Cxcl11) and co-stimulation (Cd80, Cd40) (FIG. 50). In summary, TRAIL blockade in the TME using the engineered oncolytic virus HSV-1^{α-TRAIL} boosted tumor-specific T-cell and myeloid immune responses. Overall, we established a therapeutic strategy for GBM by use of engineered oncolytic viruses targeting a specific astrocyte subset and its interaction with T cells (FIG. 5p).

TABLE 2
HSV-1 construct characteristics.

| | Parental | cDNA | miR-T | Relevant |
|---|---|---|---|---|
| Virus | strain | payload^{a} | attenuation^{b} | genotype^{c} |
| ONCR- | KOS | N/A | ICP27- | gC-EGFR fusion |
| 142 | | (Empty) | T1001 | gB-D285N, |
| | | | ICP34.5- | A549T |
| | | | T1002 | US9-R58Stop |
| | | | ICP4- | DITR (duplicate |
| | | | T1003 | copies of ICP0, |
| | | | | ICP4, ICP34.5, |
| | | | | oriS, and |
| | | | | pac deleted) |
| | | | | ICP47-DM1-S2 |
| | | | | UL3-cDNA |
| | | | | cassette-UL4 |
| | | | | UL37-loxP-UL38 |
| ONCR- | ONCR- | CAGp- | | |
| 303 | 142 | anti- | | |
| | | TRAIL | | |

^{a}CAGp, hybrid promoter containing the CMV Immediate Early enhancer element, the chicken ACTB promoter including the first exon and intron with enhancer element, and the splice acceptor site from the first intron of the rabbit HBB gene.
^{b}HSV gene with the corresponding miR-T cassette from Table 3 inserted into the 3′-UTR.
^{c}The development of the ONCR-142 HSV-1 vector was previously described³⁹.

TABLE 3
miRNA attenuation cassettes.

| Cassette | miR targets | Cell type or tissue protected |
|---|---|---|
| T1001 | miR-219a-5p | Neurons |
| | miR-122-5p | Hepatocytes |
| | miR-128T-M | Neurons |
| T1002 | miR-128T-M | Neurons |
| | miR-204 | Spinal Cord |
| | miR-219-3p | Neurons |
| T1003 | miR-124-3p | Neurons |
| | miR-1-3p | Skeletal/Cardiac Muscle |
| | miR-143-3p | Smooth Muscle |

### REFERENCES

1. Rajaratnam, V., et al., Glioblastoma: Pathogenesis and Current Status of Chemotherapy and Other Novel Treatments. Cancers, 2020. 12 (4): p. 937.
2. Stupp, R., et al., Effects of radiotherapy with concomitant and adjuvant temozolomide versus radiotherapy alone on survival in glioblastoma in a randomised phase III study: 5-year analysis of the EORTC-NCIC trial. The Lancet Oncology, 2009. 10 (5): p. 459-466.
3. Tamimi, A. F., M. Juweid, and S. De Vleeschouwer, Epidemiology and Outcome of Glioblastoma, in Glioblastoma. 2017, Codon Publications. p. 143-153.
4. Andersen, B. M., et al., Glial and myeloid heterogeneity in the brain tumour microenvironment. Nature Reviews Cancer, 2021. 21 (12): p. 786-802.
5. Quail, D. F. and J. A. Joyce, The Microenvironmental Landscape of Brain Tumors. Cancer Cell, 2017. 31 (3): p. 326-341.
6. Klemm, F., et al., Interrogation of the Microenvironmental Landscape in Brain Tumors Reveals Disease-Specific Alterations of Immune Cells. Cell, 2020. 181 (7): p. 1643-1660.e17.
7. Friebel, E., et al., Single-Cell Mapping of Human Brain Cancer Reveals Tumor-Specific Instruction of Tissue-Invading Leukocytes. Cell, 2020. 181 (7): p. 1626-1642.e20.
8. Ochocka, N., et al., Single-cell RNA sequencing reveals functional heterogeneity of glioma-associated brain macrophages. Nature Communications, 2021. 12 (1): p. 1151.
9. Pombo Antunes, A. R., et al., Single-cell profiling of myeloid cells in glioblastoma across species and disease stage reveals macrophage competition and specialization. Nature Neuroscience, 2021. 24 (4): p. 595-610.
10. Darmanis, S., et al., Single-Cell RNA-Seq Analysis of Infiltrating Neoplastic Cells at the Migrating Front of Human Glioblastoma. Cell Reports, 2017. 21 (5): p. 1399-1410.
11. Sanmarco, L. M., et al., Gut-licensed IFNγ+ NK cells drive LAMP1+TRAIL+ anti-inflammatory astrocytes. Nature, 2021. 590 (7846): p. 473-479.
12. Wheeler, M. A., et al., MAFG-driven astrocytes promote CNS inflammation. Nature, 2020. 578 (7796): p. 593-599.
13. Lee, H. G., M. A. Wheeler, and F. J. Quintana, Function and therapeutic value of astrocytes in neurological diseases. Nat Rev Drug Discov, 2022. 21 (5): p. 339-358.
14. Clark, I. C., et al., Barcoded viral tracing of single-cell interactions in central nervous system inflammation. Science, 2021. 372 (6540): p. eabf1230.
15. Sanmarco, L. M., et al., Functional immune cell-astrocyte interactions. J Exp Med, 2021. 218 (9).
16. Heiland, D. H., et al., Tumor-associated reactive astrocytes aid the evolution of immunosuppressive environment in glioblastoma. Nature Communications, 2019. 10 (1): p. 2541.
17. Mega, A., et al., Astrocytes enhance glioblastoma growth. Glia, 2020. 68 (2): p. 316-327.
18. Okolie, O., et al., Reactive astrocytes potentiate tumor aggressiveness in a murine glioma resection and recurrence model. Neuro-Oncology, 2016. 18 (12): p. 1622-1633.
19. Kim, J.-K., et al., Tumoral RANKL activates astrocytes that promote glioma cell invasion through cytokine signaling. Cancer Letters, 2014. 353 (2): p. 194-200.
20. Loeffler, S., et al., Interleukin-6 induces transcriptional activation of vascular endothelial growth factor (VEGF) in astrocytesin vivo and regulates VEGF promoter activity in glioblastoma cells via direct interaction between STAT3 and Sp1. International Journal of Cancer, 2005. 115 (2): p. 202-213.
21. Wurm, J., et al., Astrogliosis Releases Pro-Oncogenic Chitinase 3-Like 1 Causing MAPK Signaling in Glioblastoma. Cancers, 2019. 11 (10): p. 1437.
22. Couturier, C. P., et al., Glioblastoma scRNA-seq shows treatment-induced, immune-dependent increase in mesenchymal cancer cells and structural variants in distal neural stem cells. Neuro-Oncology, 2022: p. noac085.
23. Iizuka, Y., et al., Identification of a glioma antigen, GARC-1, using cytotoxic T lymphocytes induced by HSV cancer vaccine. Int J Cancer, 2006. 118 (4): p. 942-9.
24. Wang, L. B., et al., Proteogenomic and metabolomic characterization of human glioblastoma. Cancer Cell, 2021. 39 (4): p. 509-528 e20.
25. Murphy, G. M., Jr., et al., Expression of interleukin-11 and its encoding mRNA by glioblastoma cells. Neurosci Lett, 1995. 196 (3): p. 153-6.
26. Li, J., et al., PI3Kgamma inhibition suppresses microglia/TAM accumulation in glioblastoma microenvironment to promote exceptional temozolomide response. Proc Natl Acad Sci USA, 2021. 118 (16).
27. Lawler, S. E., et al., Oncolytic Viruses in Cancer Treatment: A Review. JAMA Oncol, 2017. 3 (6): p. 841-849.
28. Ma, R., Li, Z., Chiocca, E. A., Caligiuri, M. A. & Yu, J. The emerging field of oncolytic virus-based cancer immunotherapy. Trends Cancer 9, 122-139, doi: 10.1016/j.trecan.2022.10.003 (2023).
29. Peruzzi, P. & Chiocca, E. A. Viruses in cancer therapy—from benchwarmers to quarterbacks. Nat Rev Clin Oncol 15, 657-658, doi: 10.1038/s41571-018-0077-0 (2018).
30. Bischoff, J. R. et al. An adenovirus mutant that replicates selectively in p53-deficient human tumor cells. Science 274, 373-376, doi: 10.1126/science.274.5286.373 (1996).
31. Martuza, R. L., Malick, A., Markert, J. M., Ruffner, K. L. & Coen, D. M. Experimental therapy of human glioma by means of a genetically engineered virus mutant. Science 252, 854-856, doi: 10.1126/science. 1851332 (1991).
32. Shalhout, S. Z., Miller, D. M., Emerick, K. S. & Kaufman, H. L. Therapy with oncolytic viruses: progress and challenges. Nat Rev Clin Oncol 20, 160-177, doi: 10.1038/s41571-022-00719-w (2023).
33. Breitbach, C. J. et al. Intravenous delivery of a multi-mechanistic cancer-targeted oncolytic poxvirus in humans. Nature 477, 99-102, doi: 10.1038/nature10358 (2011).
34. Kennedy, E. M. et al. Design of an Interferon-Resistant Oncolytic HSV-1 Incorporating Redundant Safety Modalities for Improved Tolerability. Mol Ther Oncolytics 18, 476-490, doi: 10.1016/j.omto.2020.08.004 (2020).
35. Habib, N. et al. Massively parallel single-nucleus RNA-seq with DroNc-seq. Nat Methods 14, 955-958, doi: 10.1038/nmeth.4407 (2017).
36. Macosko, Evan Z. et al. Highly Parallel Genome-wide Expression Profiling of Individual Cells Using Nanoliter Droplets. Cell 161, 1202-1214, doi: 10.1016/j.cell.2015.05.002 (2015).
37. Takenaka, M. C. et al. Control of tumor-associated macrophages and T cells in glioblastoma via AHR and CD39. Nat Neurosci 22, 729-740, doi: 10.1038/s41593-019-0370-y (2019).
38. Lee, Y., Messing, A., Su, M. & Brenner, M. GFAP promoter elements required for region-specific and astrocyte-specific expression. Glia 56, 481-493, doi: 10.1002/glia.20622 (2008).
39. Gutierrez-Vazquez, C. & Quintana, F. J. Protocol for in vitro analysis of pro-inflammatory and metabolic functions of cultured primary murine astrocytes. STAR Protoc 3, 101033, doi: 10.1016/j.xpro.2021.101033 (2022).
40. Natarajan, R. et al. STAT3 modulation to enhance motor neuron differentiation in human neural stem cells. PLOS ONE 9, e100405, doi: 10.1371/journal.pone.0100405 (2014).
41. Bray, N. L., Pimentel, H., Melsted, P. & Pachter, L. Near-optimal probabilistic RNA-seq quantification. Nat Biotechnol 34, 525-527, doi: 10.1038/nbt.3519 (2016).
42. Frankish, A. et al. Gencode 2021. Nucleic Acids Res 49, D916-D923, doi: 10.1093/nar/gkaa1087 (2021).
43. Soneson, C., Love, M. I. & Robinson, M. D. Differential analyses for RNA-seq: transcript-level estimates improve gene-level inferences. F1000Res 4, 1521, doi: 10.12688/f1000research.7563.2 (2015).
44. Chu, T., Wang, Z., Pe′er, D. & Danko, C. G. Cell type and gene expression deconvolution with BayesPrism enables Bayesian integrative analysis across bulk and single-cell RNA sequencing in oncology. Nat Cancer 3, 505-517, doi: 10.1038/s43018-022-00356-3 (2022).
45. Badia, I. M. P. et al. decoupleR: ensemble of computational methods to infer biological activities from omics data. Bioinform Adv 2, vbac016, doi: 10.1093/bioadv/vbac016 (2022).
46. Tang, Z., Kang, B., Li, C., Chen, T. & Zhang, Z. GEPIA2: an enhanced web server for large-scale expression profiling and interactive analysis. Nucleic Acids Res 47, W556-W560, doi: 10.1093/nar/gkz430 (2019).
47. Durafourt, B. A., Moore, C. S., Blain, M. & Antel, J. P. Isolating, culturing, and polarizing primary human adult and fetal microglia. Methods Mol Biol 1041, 199-211, doi: 10.1007/978-1-62703-520-0_19 (2013)

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An engineered herpes simplex viruses (oHSV) comprising a sequence encoding a tumor necrosis factor-related apoptosis-inducing ligand (TRAIL)-blocking monoclonal antibody or antigen-binding fragment thereof.

2. The oHSV of claim 1, further comprising one or more mutations that maximize infection and replication rates via fusogenic mutations.

3. The oHSV of claim 1, further comprising one or more microRNA target sites (miRTS) into the virus.

4. The oHSV of claim 3, wherein the one or more miRNAs are selected from miR-124, miR-1, miR-143, miR-128, miR-219, miR-219a, miR-122, miR-204, miR-217, miR-137, miR-142, and miR-126.

5. The oHSV of claim 1, further comprising one or more mutations in UL37 that inhibit retrograde axonal transport.

6. The oHSV of claim 1, further comprising one or more deletions in the genes encoding viral ICP6 and ICP34.5 that reduce replication in non-replicating cells.

7. The oHSV of claim 1, wherein the antigen-binding fragment thereof is an scFv, scAb, Fab, or Fab₂.

8. The oHSV of claim 1, wherein the oncolytic virus further comprises one or more additional transgenes.

9. The oHSV of claim 8, wherein the one or more additional transgenes comprise sequences encoding one, two, or more of interleukin-12 (IL-12), FLT3LG (extracellular domain), CCL4, and/or antibodies to immune checkpoints PD-1 and/or CTLA-4.

10. A method for the prevention, treatment, and/or amelioration of a cancer in a subject, the method comprising administering a therapeutically effective amount of an oncolytic virus of claim 1, to a subject in need thereof.

11. The method of claim 10, wherein the cancer is brain cancer.

12. The method of claim 11, wherein the brain cancer is glioblastoma.

13. (canceled)

14. (canceled)

15. (canceled)

16. The method of claim 10, further comprising one or more mutations that maximize infection and replication rates via fusogenic mutations.

17. The method of claim 10, further comprising one or more microRNA target sites (miRTS) into the virus.

18. The method of claim 17, wherein the one or more miRNAs are selected from miR-124, miR-1, miR-143, miR-128, miR-219, miR-219a, miR-122, miR-204, miR-217, miR-137, miR-142, and miR-126.

19. The method of claim 10, further comprising one or more mutations in UL37 that inhibit retrograde axonal transport.

20. The method of claim 10, further comprising one or more deletions in the genes encoding viral ICP6 and ICP34.5 that reduce replication in non-replicating cells.

21. The method of claim 10, wherein the antigen-binding fragment thereof is an scFv, scAb, Fab, or Fab₂.

22. The method of claim 10, wherein the oncolytic virus further comprises one or more additional transgenes.

23. The method of claim 22, wherein the one or more additional transgenes comprise sequences encoding one, two, or more of interleukin-12 (IL-12), FLT3LG (extracellular domain), CCL4, and/or antibodies to immune checkpoints PD-1 and/or CTLA-4.


SEQUENCE LISTING (15 sequences)

SEQ ID NO: 1 - protein, 442 aa, synthetic construct
QVTLKESGPGILQPSQTLSLTCTFSGFSLTTHGMGVGWIRQPAGKGLEWLANIWWDDDKFHSPSLKNRLTISKDTSNNQAFLRITNVDTADAAIYYCARISWERAYYFDYWGQGVMVTVSSAETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPG

SEQ ID NO: 2 - protein, 213 aa, synthetic construct
DVQMTQSPSFLSASVGERVTLSCRASQNVDTYLDWYQQKLGEAPKLLIYNTNNLHTGIPSRFSGSGSGTDYTLTISSLQPEDVATYFCLQRNRWLTFGSGTKLEIKRADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 3 - DNA, 82 bp, synthetic construct
  source 1..82: mol_type = other DNA
tttttttaagcagtggtatcaacgcagagtacnnnnnnnnnnnnnnnnnnnntttttttttttttttttttttttttttttt

SEQ ID NO: 4 - DNA, 33 bp, synthetic construct
  source 1..33: mol_type = other DNA
aagcagtggtatcaacgcagagtgaatrgrgrg

SEQ ID NO: 5 - DNA, 23 bp, synthetic construct
  source 1..23: mol_type = other DNA
aagcagtggtatcaacgcagagt

SEQ ID NO: 6 - DNA, 66 bp, synthetic construct
  source 1..66: mol_type = other DNA
aatgatacggcgaccaccgagatctacacgcctgtccgcggaagcagtggtatcaacgcagagtac

SEQ ID NO: 7 - DNA, 37 bp, synthetic construct
  source 1..37: mol_type = other DNA
gcctgtccgcggaagcagtggtatcaacgcagagtac

SEQ ID NO: 8 - DNA, 23 bp, synthetic construct
  source 1..23: mol_type = other DNA
aaaggcgcgccgagggcctattt

SEQ ID NO: 9 - DNA, 33 bp, synthetic construct
  source 1..33: mol_type = other DNA
ttttttggtctcccggtgtttcgtcctttccac

SEQ ID NO: 10 - DNA, 25 bp, synthetic construct
  source 1..25: mol_type = other DNA
gttccctgcaggaaaaaagcaccga

SEQ ID NO: 11 - DNA, 43 bp, synthetic construct
  source 1..43: mol_type = other DNA
  misc_feature 18: note = repeat_unit 1 to 20
aaaaaaggtctctaccgngttttagagctagaaatagcaagtt

SEQ ID NO: 12 - DNA, 20 bp, synthetic construct
  source 1..20: mol_type = other DNA
gcactaccagagctaactca

SEQ ID NO: 13 - DNA, 20 bp, synthetic construct
  source 1..20: mol_type = other DNA
acttctctccacccagctgc

SEQ ID NO: 14 - DNA, 20 bp, synthetic construct
  source 1..20: mol_type = other DNA
ttcatagtctaccgcttggc

SEQ ID NO: 15 - DNA, 20 bp, synthetic construct
  source 1..20: mol_type = other DNA
gcagccattgtacatgccgg